(19) United States
(12) Patent Application Publication
(10) Pub. No.: US 2024/0238258 A1
(43) Pub. Date: Jul. 18, 2024

(54) INHIBITORS OF PROTEIN TYROSINE PHOSPHATASE, COMPOSITIONS, AND METHODS OF USE

(71) Applicant: BRISTOL-MYERS SQUIBB COMPANY, PRINCETON, NJ (US)

(72) Inventors: HAIBO LIU, LEXINGTON, MA (US); YUCHENG MU, SOMERVILLE, MA (US); ANNAPURNA PENDRI, SOUTH GLASTONBURY, CT (US); SHOSHANA L. POSY, HIGHLAND PARK, NJ (US); JOANNE JEWETT BRONSON, WENHAM, MA (US); LOUIS S. CHUPAK, OLD SAYBROOK, CT (US); LAURA AKULLIAN D'AGOSTINO, SUDBURY, MA (US)

(73) Assignee: BRISTOL-MYERS SQUIBB COMPANY, PRINCETON, NJ (US)

(21) Appl. No.: 18/389,887

(22) Filed: Dec. 20, 2023

Related U.S. Application Data
(60) Provisional application No. 63/476,513, filed on Dec. 21, 2022.

(51) Int. Cl. A61K 31/433; A61K 39/395; C07D 417/12

(52) U.S. Cl. CPC A61K 31/433; A61K 39/3955; C07D 417/12

(57) ABSTRACT

Disclosed are compounds of Formula (I)

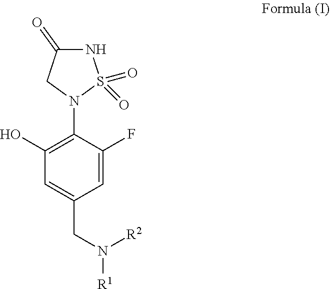

pharmaceutically acceptable salts thereof are defined herein, and pharmaceutical compositions thereof and combinations thereof, and methods of using the same as inhibitors of protein tyrosine phosphatases (PTPN2). These compounds are useful in treating cancer and diseases susceptible to PTPN2 inhibition.

### CROSS REFERENCE TO RELATED APPLICATIONS

This application claims the benefit of U.S. Provisional Application Ser. No. 63/476,513 filed Dec. 21, 2022 which is incorporated herein in its entirety.

### FIELD OF THE INVENTION

Disclosed are compounds, pharmaceutically acceptable salts thereof, pharmaceutical compositions thereof and combinations thereof, and methods of using the same as inhibitors of protein tyrosine phosphatases.

### BACKGROUND

Immune checkpoint blockade (ICB) is an innovative approach to immunotherapy that targets immune evasion mechanisms to improve clinical responses in cancer patients. For example, checkpoint blockade antibodies target cytotoxic T lymphocyte antigen 4 (CTLA-4), programmed cell death 1 (PD-1), and its ligands, such as programmed cell death ligand 1 (PD-L1), in the treatment of multiple types of cancer to significantly improve the treatment and survival outcomes of patients affected by these malignancies.

A majority of patients who undergo ICB, however, are either refractory to treatment or eventually acquire resistance. In particular, mutation or loss of interferon-gamma (IFNγ) signaling pathway represents a significant mechanism of clinical ICB resistance (Zaretsky, N. Engl. J. Med. 375, 819-829). IFNγ is a T-cell-derived cytokine that signals through the Janus kinase/signal transducer and activator of transcription pathway (JAK/STAT) to restrict tumor growth directly. Furthermore, IFNγ indirectly restricts tumor growth by promoting upregulation of major histocompatibility complex class I (MHC-I), thereby enabling antigen (Ag) presentation to T-cells. In vivo CRISPR screening using syngeneic mouse models has revealed enrichment of the IFNγ pathway in tumors resistant to anti-PD-1. These studies identified the aforementioned IFNγ pathway members (JAK1/2 and STAT1) and Interferon Gamma Receptor (IFNGR1/IFNGR2) as resistance hits, in addition to newly identified negative regulators—such as PTPN2 and Apelin Receptor (APLNR)—which represent novel therapeutic targets (Charles Sinclair et al. Emerg Top Life Sci. (2021) 5 (5): 675-680).

Data pooled from in vivo genetic screening using CRISPR-Cas9 genome editing to identify genes that cause resistance to checkpoint blockade identified that deletion of the protein tyrosine phosphatase (PTPN2) gene in tumor cells increased the efficacy of immunotherapy. The PTPN2 gene encodes a protein tyrosine phosphatase that regulates a range of intracellular processes. Loss of PTPN2 in tumor cells promotes amplified IFNγ signaling, antigen presentation to T cells and growth arrest in response to cytokines; these data suggest that PTPN2 therapeutic inhibition may potentiate the effect of immunotherapies that invoke an IFNγ response (Manguso, Robert T et al. Nature vol. 547, 7664 (2017): 413-418).

Protein tyrosine phosphatase non-receptor type 2 (PTPN2), also known as T cell protein tyrosine phosphatase (TCPTP), is an intracellular member of the class 1 subfamily phospho-tyrosine specific phosphatases that control multiple cellular regulatory processes by removing phosphate groups from tyrosine substrates. PTPN2 is ubiquitously expressed, but expression is highest in hematopoietic and placental cells (Mosinger, B. Jr. et al., Proc Natl Acad Sci USA (1992) 89:499-503). In humans, PTPN2 expression is controlled post-transcriptionally by the existence of two splice variants: a 45 kDa form that contains a nuclear localization signal at the C-terminus upstream of the splice junction and a 48 kDa canonical form which has a C-terminal ER retention motif (Tillmann U. et al., Mol Cell Biol (1994) 14:3030-3040). The 45 kDa isoform can passively transfuse into the cytosol under certain cellular stress conditions. Both isoforms share an N-terminal phospho-tyrosine phosphatase catalytic domain, and as a critical negative regulator of the JAK-STAT pathway, PTPN2 directly regulates signaling through cytokine receptors. The PTPN2 catalytic domain shares 74% sequence homology with PTPN1 (also called PTP1B) and shares similar enzymatic kinetics (Romsicki Y. et al., Arch Biochem Biophys (2003) 414:40-50).

T cell protein tyrosine phosphatase PTPN2 has been further identified as a key negative regulator of TCR signaling, underscoring an association between PTPN2 Single nucleotide polymorphismts (SNPs) and autoimmune disease (Wiede F et al., J Clin Invest. (2011); 121(12):4758-4774). PTPN2 dephosphorylates and inactivates Src family kinases to regulate T cell responses. PTPN2 deficiency has been demonstrated to lower the in vivo threshold for TCR-dependent CD8⁺ T cell proliferation. Consistent with these findings, T cell-specific PTPN2-deficient mice have been shown to develop widespread inflammation and autoimmunity. This autoimmunity is associated with increased serum levels of proinflammatory cytokines, anti-nuclear antibodies, T cell infiltrates in non-lymphoid tissues, and liver disease. These data further indicate that PTPN2 is a critical negative regulator of TCR signaling that sets the threshold for TCR-induced naive T cell responses to prevent autoimmune and inflammatory disorders.

In addition to PTPN2 encoding T cell PTP (TCPTP) as a susceptibility locus for autoimmune diseases, SNPs in PTPN2 have been linked to the development of type 1 diabetes, rheumatoid arthritis, and Crohn's disease. Moreover, a type 1 diabetes-linked PTPN2 variant rs1893217(C) has also been associated with decreased PTPN2 expression in T cells (Florian Wiede J Clin Invest. 2011; 121(12):4758-4774)

The above findings suggest that inhibition of PTPN2 is a potential therapeutic strategy to improve the efficacy of cancer therapy regimens associated with ICB resistance.

### SUMMARY

The present disclosure is directed to compounds pharmaceutically acceptable salts thereof, pharmaceutical compositions thereof, and combinations thereof, are effective inhibitors of protein tyrosine phosphatases, e.g., protein tyrosine phosphatase non-receptor type 2 (PTPN2) and/or protein tyrosine phosphatase non-receptor type 1 ((PTPN1), also known as protein tyrosine phosphatase-1B (PTP1B)). The invention further provides methods of treating, preventing, or ameliorating cancers comprising administering to a subject in need thereof an effective amount of PTPN2/PTPN1 inhibitors disclosed herein. In a preferred embodiment, the compounds have a mono-cyclic core structure compared to literature-reported compounds, where compounds contain fused bicyclic cores.

In some embodiments, disclosed herein is an inhibitor of protein tyrosine phosphatase, e.g., PTPN2 and/or PTP1B, comprising a compound disclosed herein, e.g., a compound of Formula (I). In other embodiments, disclosed herein are methods of treating a disease or disorder, e.g., cancer, type-2 diabetes, obesity, a metabolic disease, or any other disease, disorder or ailment favorably responsive to PTPN2 or PTP1B inhibitor treatment, comprising administering an effective amount of a compound disclosed herein, e.g., a compound of Formula (I). These and other features of the invention will be set forth in expanded form in this disclosure.

The first aspect of the present invention provides at least one compound of Formula (I) of the following structure:

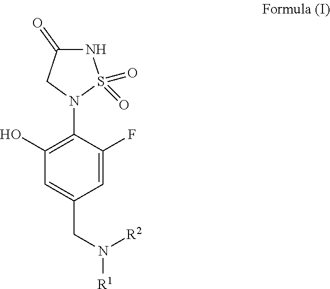

wherein, independently for each occurrence:
R¹ is selected from the group consisting of: 1H-1,2,4-triazol-3-yl,

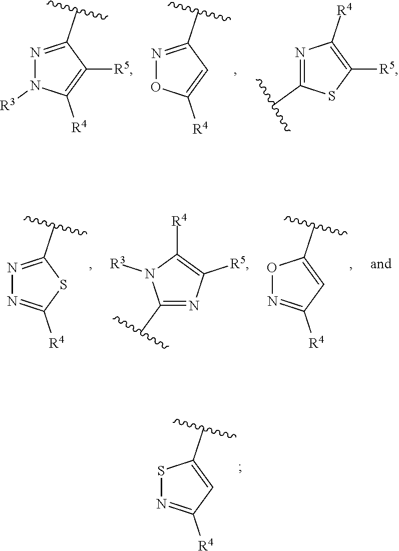

R² is selected from the group consisting of: —H, alkyl and substituted alkyl;
R³ is selected from the group consisting of: —H, alkyl and substituted alkyl;
R⁴ is selected from the group consisting of: —H, alkyl, substituted alkyl, branched alkyl, alkoxy, halogen, cyano, amine, hydroxy, aryl and substituted aryl;
R⁵ is selected from the group consisting of: —H, alkyl, substituted alkyl, branched alkyl, alkoxy, halogen, cyano, amine, hydroxy, aryl and substituted aryl.

Further disclosed is a compound selected from a group consisting of:
5-(4-(((4-(2,4-dimethylphenyl)thiazol-2-yl)(methyl)amino)methyl)-2-fluoro-6-hydroxyphenyl) -1,2,5-thiadiazolidin-3-one 1,1-dioxide;
5-(4-(((1H-pyrazol-3-yl)amino)methyl)-2-fluoro-6-hydroxyphenyl)-1,2,5-thiadiazolidin-3-one 1,1-dioxide;
5-(2-fluoro-6-hydroxy-4-((isoxazol-3-ylamino)methyl)phenyl)-1,2,5-thiadiazolidin-3-one 1,1dioxide;
5-(2-fluoro-6-hydroxy-4-(((5-methylisoxazol-3-yl)amino)methyl)phenyl)-1,2,5-thiadiazolidin-3-one 1,1-dioxide;
5-(4-(((1H-1,2,4-triazol-3-yl)amino)methyl)-2-fluoro-6-hydroxyphenyl)-1,2,5-thiadiazolidin-3-one 1,1-dioxide;
5-(2-fluoro-6-hydroxy-4-(((1-methyl-1H-pyrazol-3-yl)amino)methyl)phenyl)-1,2,5-thiadiazolidin-3-one 1,1-dioxide;
5-(4-(((1,3,4-thiadiazol-2-yl)amino)methyl)-2-fluoro-6-hydroxyphenyl)-1,2,5-thiadiazolidin-3-one 1,1-dioxide;
5-(2-fluoro-6-hydroxy-4-(((5-methyl-1H-imidazol-2-yl)amino)methyl)phenyl)-1,2,5-thiadiazolidin-3-one 1,1-dioxide;
5-(2-fluoro-6-hydroxy-4-((thiazol-2-ylamino)methyl)phenyl)-1,2,5-thiadiazolidin-3-one 1,1-dioxide;
5-(4-(((1H-imidazol-2-yl)amino)methyl)-2-fluoro-6-hydroxyphenyl)-1,2,5-thiadiazolidin-3-one 1,1-dioxide;
5-(2-fluoro-6-hydroxy-4-(((1-methyl-1H-imidazol-2-yl)amino)methyl)phenyl)-1,2,5-thiadiazolidin-3-one 1,1-dioxide;
5-(2-fluoro-6-hydroxy-4-(((5-methyl-1,3,4-thiadiazol-2-yl)amino)methyl)phenyl)-1,2,5-thiadiazolidin-3-one 1,1-dioxide;
5-(2-fluoro-6-hydroxy-4-(((4-methylthiazol-2-yl)amino)methyl)phenyl)-1,2,5-thiadiazolidin-3 one 1,1-dioxide;
5-(2-fluoro-6-hydroxy-4-(((3-methylisothiazol-5-yl)amino)methyl)phenyl)-1,2,5-thiadiazolidin 3-one 1,1-dioxide;
5-(2-fluoro-6-hydroxy-4-(((3-methyl-1H-pyrazol-5-yl)amino)methyl)phenyl)-1,2,5-thiadiazolidin-3-one 1,1-dioxide; or pharmaceutically acceptable salts thereof.

In some embodiments, the compound of Formula (I) is formulated as a pharmaceutically acceptable composition comprising the compound of Formula (I) and a pharmaceutically acceptable carrier.

Also disclosed herein is a method of treating cancer in a patient in need thereof, comprising administering to the patient an effective amount of the compound of formula (I) disclosed herein in combination with an additional therapeutic agent. In some embodiments, the additional therapeutic agent is an immunotherapeutic agent. For example, in some embodiments, the immunotherapeutic agent is an antibody.

Also disclosed herein is a method of treating cancer in a patient in need thereof, comprising administering to the patient an effective amount of a compound disclosed herein, e.g., a compound of Formula (I).

Further disclosed herein is a method of treating a metabolic disease in a patient in need thereof, comprising administering to the patient an effective amount of a compound disclosed herein, e.g., a compound of Formula (I).

In some embodiments, the method comprises the treatment of cancer. In some embodiments, the cancer comprises pancreatic cancer, breast cancer, multiple myeloma, melanoma, or a cancer of the secretory cells.

Also disclosed herein is a composition for use in treating cancer in a patient in need thereof, wherein the composition comprises a compound disclosed herein, e.g., a compound of Formula (I) in combination with an additional therapeutic agent. In some embodiments, the additional therapeutic agent is an immunotherapeutic agent. For example, in some embodiments, the immunotherapeutic agent is selected from the group consisting of an anti-PD-1 antibody, and an anti-PD-L1 antibody.

Further disclosed herein is a composition for use in treating a metabolic disease in a patient in need thereof, wherein the composition comprises a compound disclosed herein, e.g., a compound of Formula (I).

### DETAILED DESCRIPTION

The present disclosure is directed to compounds pharmaceutically acceptable salts thereof, pharmaceutical compositions thereof, and combinations thereof, are effective inhibitors of protein tyrosine phosphatases, e.g., protein tyrosine phosphatase non-receptor type 2 (PTPN2) and/or protein tyrosine phosphatase non-receptor type 1 ((PTPN1), also known as protein tyrosine phosphatase-1B (PTP1B)). The invention further provides methods of treating, preventing, or ameliorating cancers comprising administering to a subject in need thereof an effective amount of PTPN2/PTPN1 inhibitors disclosed herein. In a preferred embodiment, the compounds have a mono-cyclic core structure compared to literature-reported compounds, where compounds contain fused bicyclic cores.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

Compounds described herein can comprise one or more asymmetric centers, and thus can exist in various isomeric forms, e.g., enantiomers and/or diastereomers. For example, the compounds described herein can be in the form of an individual enantiomer, diastereomer, geometric isomer, or a mixture of stereoisomers, including racemic mixtures and mixtures enriched in one or more stereoisomers. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972) The disclosure additionally encompasses compounds described herein as individual isomers substantially free of other isomers, and alternatively, as mixtures of various isomers.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound.

The features and advantages of the invention as described in this disclosure may be more readily understood by those of ordinary skill in the art in view of the following definitions. Certain features of the invention described within the context of separate embodiments may also be combined to form a single or extrapolated to include multiple embodiments. Embodiments identified herein as exemplary or preferred are illustrative and not limiting.

Unless expressly stated otherwise herein, references made in the singular may also include the plural. For example, “a” and “an” may refer to either one or one or more.

As used herein, the phrase “compounds” refers to at least one compound. For example, a compound of Formula (I) includes a compound of Formula (I) and two or more compounds of Formula (I).

Unless otherwise indicated, any heteroatom with unsatisfied valences is assumed to have hydrogen atoms sufficient to satisfy the valences.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

Throughout the specification, groups and substituents thereof may be chosen by one skilled in the field to provide stable moieties and compounds.

In accordance with a convention used in the art,

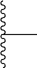

is used in structural formulas herein to depict the bond that is the point of attachment of the moiety
or substituent to the core or backbone structure.

The terms “halo” and “halogen,” as used herein, refer to F, Cl, Br, and I.

The term “cyano” refers to the group —CN.

The term “amino” refers to the group —NH₂.

The term “oxo” refers to the group ═O.

The term “alkyl” as used herein, refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₆ alkyl” denotes straight and branched chain alkyl groups with one to six carbon atoms.

The term “fluoroalkyl” as used herein is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “cyanoalkyl” includes both branched and straight-chain saturated alkyl groups substituted with one or more cyano groups. For example, “cyanoalkyl” includes —CH₂CN, —CH₂CH₂CN, and C₁₋₄ cyanoalkyl.

The term “aminoalkyl” includes both branched and straight-chain saturated alkyl groups substituted with one or more amine groups. For example, “aminoalkyl” includes —CH₂NH₂, —CH₂CH₂NH₂, and C₁₋₄ aminoalkyl.

The term “hydroxyalkyl” includes both branched and straight-chain saturated alkyl groups substituted with one or more hydroxyl groups. For example, “hydroxyalkyl” includes —CH₂OH, —CH₂CH₂OH, and C₁₋₄ hydroxyalkyl.

The term “hydroxy-fluoroalkyl” includes both branched and straight-chain saturated alkyl groups substituted with one or more hydroxyl groups and one or more fluorine atoms. For example, “hydroxy-fluoroalkyl” includes —CHFCH₂OH, —CH₂CHFC(CH₃)₂OH, and C₁₋₄ hydroxy-fluoroalkyl.

The term “cycloalkyl,” “carbocyclic” “carbocyclyl” as used herein, refers to a group derived from a non-aromatic monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃-C₆ cycloalkyl” denotes cycloalkyl groups with three to six carbon atoms.

The term “heterocyclic” as used herein, refers to organic compounds with cyclic structures of both carbon atoms and non-carbon atoms such as oxygen, nitrogen.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The term “alkoxyalkyl,” as used herein, refers to an alkoxy group attached through its oxygen atom to an alkyl group, which is attached to the parent molecular moiety, for example, methoxymethyl group (—CH₂OCH₃). For example, “C₂₋₄ alkoxyalkyl” denotes alkoxyalkyl groups with two to four carbon atoms, such as —CH₂OCH₃, —CH₂CH₂OCH₃, —CH₂OCH₂CH₃, and —CH₂CH₂OCH₂CH₃.

The term “amine” or “amines” as used herein refers to compounds in which a nitrogen atom is directly bonded to several carbon atoms. Embodiments are comprised of derivatives of ammonia (—NH₃) resulting from a progressive substitution of the three hydrogen atoms by hydrocarbon groups. Amines are classified as primary, secondary, or tertiary by the number of carbons bonded to the nitrogen atom. For example, a primary amine has one carbon bonded to the nitrogen (R—NH₂), a secondary amine has two carbons bonded to the nitrogen, amine (R2-NH), and a tertiary amine has three carbons bonded to the nitrogen (R3-N) wherein R is an alkyl group.

The term “heteroaryl” as used herein, refers to an aromatic heterocycle ring of 5 to 10 members and having at least one heteroatom selected from nitrogen, oxygen and sulfur, and containing at least 1 carbon atom, including both mono- and bicyclic ring systems.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (I) can be provided as amorphous solids or crystalline solids. Lyophilization can be employed to provide the compounds of Formula (I) as amorphous solids.

It should further be understood that solvates (e.g., hydrates) of the compounds of Formula (I) are also within the scope of the present invention. The term “solvate” means a physical association of a compound of Formula (I) with one or more solvent molecules, whether organic or inorganic. This physical association includes hydrogen bonding. In certain instances, the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

Various forms of prodrugs are well known in the art and are described in:
a) The Practice of Medicinal Chemistry, Camille G. Wermuth et al., Ch 31, (Academic Press, 1996);
b) Design of Prodrugs, edited by H. Bundgaard, (Elsevier, 1985);
c) A Textbook of Drug Design and Development, P. Krogsgaard-Larson and H. Bundgaard, eds. Ch 5, pgs 113-191 (Harwood Academic Publishers, 1991); and
d) Hydrolysis in Drug and Prodrug Metabolism, Bernard Testa and Joachim M. Mayer, (Wiley-VCH, 2003).

In addition, compounds of Formula (I), subsequent to their preparation, can be isolated and purified to obtain a composition containing an amount by weight equal to or greater than 99% of a compound of Formula (I) (“substantially pure”), which is then used or formulated as described herein. Such “substantially pure” compounds of Formula (I) are also contemplated herein as part of the present invention.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

A person of ordinary skill in the art would also understand that the compounds described and claimed herein as embodiments of the invention also exist in their “tautomeric forms.” As used herein, Tautomers that exist in tautomeric form pertain to compounds that are structural isomers that can readily interconvert in rapid equilibrium. As used herein the process of interconversion is called “tautomerization.”

For example, the following an embodiment a pyridone tautomer may be represented by the following:

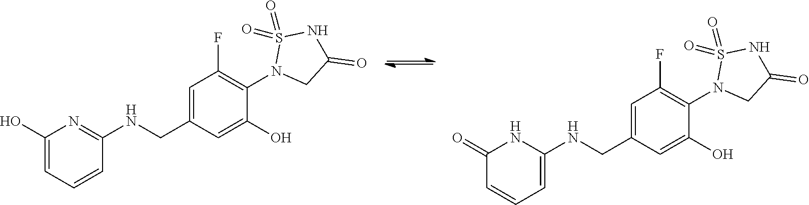 [SMILES: O=C1CN(c2c(O)cc(CNc3cccc(O)n3)cc2F)S(=O)(=O)N1 ; O=C1CN(c2c(O)cc(CNc3cccc(=O)[nH]3)cc2F)S(=O)(=O)N1]

The disclosed structures readily interconvert between left-handed and right-handed structural representations.

“Therapeutically effective amount” is intended to include an amount of a compound of the present invention alone or an amount of the combination of compounds claimed or an amount of a compound of the present invention in combination with other active ingredients effective to act as an inhibitor or effective to treat or ameliorate cancer.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

The compounds of the present invention are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. For example, methyl (—CH₃) also includes deuterated methyl groups such as -CD₃.

The term “pharmaceutically acceptable salts” is meant to include salts of active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, magnesium salt, or a similar salt.

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments, inhibition refers to a reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in the activity of a protein tyrosine phosphatase, e.g., protein tyrosine phosphatase non-receptor type 2 (PTPN2) or protein tyrosine phosphatase non-receptor type 1 (PTP1B). Thus, inhibition may include, at least in part, partially or totally decreasing stimulation, decreasing or reducing activation, or inactivating, desensitizing, or down-regulating signal transduction or enzymatic activity or the amount of a protein tyrosine phosphatase, e.g., protein tyrosine phosphatase non-receptor type 2 (PTPN2) or protein tyrosine phosphatase non-receptor type 1 (PTP1B).

“Patient” or “subject” in need thereof refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a domesticated animal. In some embodiments, a patient is a dog. In some embodiments, a patient is a parrot. In some embodiments, a patient is livestock animal. In some embodiments, a patient is a mammal In some embodiments, a patient is a cat. In some embodiments, a patient is a horse. In some embodiments, a patient is bovine. In some embodiments, a patient is a canine. In some embodiments, a patient is a feline. In some embodiments, a patient is an ape. In some embodiments, a patient is a monkey. In some embodiments, a patient is a mouse. In some embodiments, a patient is an experimental animal. In some embodiments, a patient is a rat. In some embodiments, a patient is a hamster. In some embodiments, a patient is a test animal. In some embodiments, a patient is a newborn animal. In some embodiments, a patient is a newborn human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

“Disease”, “disorder” or “condition” refers to a state of being or health status of a patient or subject capable of being treated with a compound, pharmaceutical composition, or method provided herein. In some embodiments, the compounds and methods described herein comprise reduction or elimination of one or more symptoms of the disease, disorder, or condition, e.g., through administration of a compound disclosed herein, a pharmaceutically acceptable salt thereof, or a pharmaceutical composition comprising a compound disclosed herein, or a pharmaceutically acceptable salt thereof, and a pharmaceutically acceptable carrier.

The term “signaling pathway” as used herein refers to a series of interactions between cellular and optionally extra-cellular components (e.g., proteins, nucleic acids, small molecules, ions, lipids) that conveys a change in one component to one or more other components, which in tum may convey a change to additional components, which is optionally propagated to other signaling pathway components.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's solution, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it.

Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a compound or composition described herein is administered at the same time, just prior to, or just after the administration of one or more additional therapies (e.g., anti-cancer agent, chemotherapeutic, or immunotherapeutic agent). The compounds or compositions described herein can be administered alone or can be coadministered to the patient. Coadministration is meant to include simultaneous or sequential administration of the compound or composition individually or in combination (more than one compound or agent). Thus, the preparations can also be combined, when desired, with other active substances (e.g., to reduce metabolic degradation).

Pharmaceutical compositions described herein can be prepared by any method known in the art of pharmacology. In general, such preparatory methods include the steps of bringing a disclosed compound (the “active ingredient”) into association with a carrier and/or one or more other accessory ingredients, and then, if necessary and/or desirable, shaping and/or packaging the product into a desired single- or multi-dose unit. Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

### Methods of Treatment

The present disclosure features compounds, compositions, and methods comprising a compound disclosed herein, e.g., a compound of Formula (I). In some embodiments, the compounds, compositions, and methods disclosed herein are used in the prevention or treatment of a disease, disorder, or condition. Exemplary diseases, disorders, or conditions include, but are not limited to cancer, type-2 diabetes, metabolic syndrome, obesity, or a metabolic disease.

### Cancer

In some embodiments, a compound disclosed herein, e.g., a compound of Formula (I), is used to treat cancer. As used herein, “cancer” refers to human cancers and carcinomas, sarcomas, adenocarcinomas (e.g., papillary adenocarcinomas), lymphomas, leukemias, melanomas, etc., including solid and lymphoid cancers, kidney, breast, lung, bladder, colon, ovarian, prostate, pancreas, stomach, brain, head and neck, skin, uterine, testicular, glioma, esophagus, liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), and/or multiple myeloma. In some further instances, “cancer” refers to lung cancer, breast cancer, ovarian cancer, epithelial ovarian cancer, leukemia, lymphoma, melanoma, pancreatic cancer, sarcoma, bladder cancer, bone cancer, biliary tract cancer, adrenal gland cancer, salivary gland cancer, bronchus cancer, oral cancer, cancer of the oral cavity or pharynx, laryngeal cancer, renal cancer, gynecologic cancers, brain cancer, central nervous system cancer, peripheral nervous system cancer, cancer of the hematological tissues, small bowel or appendix cancer, cervical cancer, colon cancer, esophageal cancer, gastric cancer, liver cancer, head and neck cancer, kidney cancer, myeloma, thyroid cancer, prostate cancer, metastatic cancer, or carcinoma.

Exemplary cancers that may be treated with a compound, pharmaceutical composition, or method provided herein include lymphoma, B-cell lymphoma, heavy chain disease, alpha chain disease, gamma chain disease, mu chain disease, Waldenstrom's macroglobulinemia, benign monoclonal gammopathy, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., ER-positive, ER-negative, chemotherapy-resistant, Herceptin resistant, HER² positive, doxorubicin-resistant, tamoxifen-resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, acoustic neuroma, retinoblastoma, astrocytoma, craniopharyngioma, hemangioblastoma, pinealoma, ependymoma, oligodendroglioma, meningioma, glioma, or melanoma. Additional examples include cancer of the thyroid, endocrine system, brain, breast, cervix, colon, head & neck, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, immunocytic amyloidosis, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, and hepatocellular carcinoma.

The first aspect of the present invention provides at least one compound of Formula (I) of the following structure:

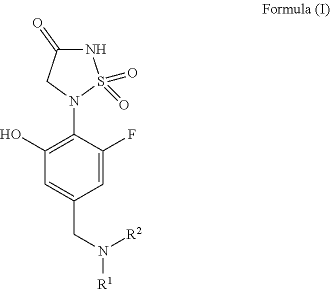

wherein, independently for each occurrence:
R¹ is selected from the group consisting of: 1H-1,2,4-triazol-3-yl,

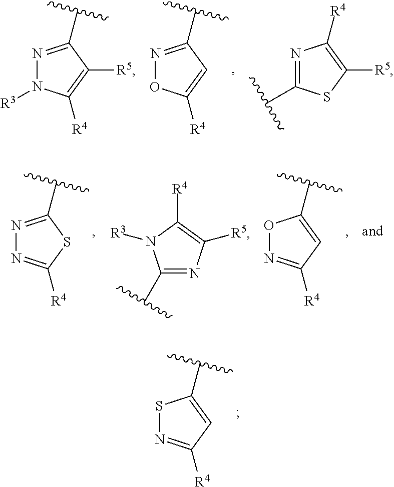

R² is selected from the group consisting of: —H, alkyl and substituted alkyl;
R³ is selected from the group consisting of: —H, alkyl and substituted alkyl;
R⁴ is selected from the group consisting of: —H, alkyl, substituted alkyl, branched alkyl, alkoxy, halogen, cyano, amine, hydroxy, aryl and substituted aryl;
R⁵ is selected from the group consisting of: —H, alkyl, substituted alkyl, branched alkyl, alkoxy, halogen, cyano, amine, hydroxy, aryl and substituted aryl.

In one embodiment of the compound of formula (I):
R¹ is selected from the group consisting of:

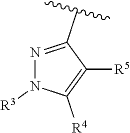

R³ is selected from the group consisting of: —H and —CH₃;
R⁴ is —H and —CH₃.

In another embodiment of the compound of formula (I): R¹ is

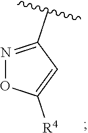

R⁴ is selected from the group consisting of: —H and —CH₃.

In one embodiment of the compound of formula (I):
R¹ is

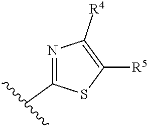

R⁴ is selected from the group consisting of: —H, —CH₃, and 2,4-dimethylphenyl;
R⁵ is selected from the group consisting of: —H and —CH₃.

In another embodiment of the compound of formula (I):
R¹ is selected from the group consisting of:

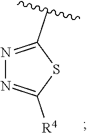

R⁴ is selected from the group consisting of: —H and —CH₃.

In one embodiment of the compound of formula (I):
R¹ is

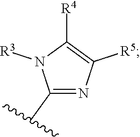

R³ is selected from the group consisting of: —H and —CH₃
R⁴ is —H and —CH₃;
R⁵ is —H and —CH₃.

In another embodiment the compounds are selected from a group consisting of:
5-(4-(((4-(2,4-dimethylphenyl)thiazol-2-yl)(methyl)amino)methyl)-2-fluoro-6-hydroxyphenyl) -1,2,5-thiadiazolidin-3-one 1,1-dioxide;
5-(4-(((1H-pyrazol-3-yl)amino)methyl)-2-fluoro-6-hydroxyphenyl)-1,2,5-thiadiazolidin-3-one 1,1-dioxide;
5-(2-fluoro-6-hydroxy-4-((isoxazol-3-ylamino)methyl)phenyl)-1,2,5-thiadiazolidin-3-one 1,1-dioxide;
5-(2-fluoro-6-hydroxy-4-(((5-methylisoxazol-3-yl)amino)methyl)phenyl)-1,2,5-thiadiazolidin-3-one 1,1-dioxide;
5-(4-(((1H-1,2,4-triazol-3-yl)amino)methyl)-2-fluoro-6-hydroxyphenyl)-1,2,5-thiadiazolidin-3-one 1,1-dioxide;
5-(2-fluoro-6-hydroxy-4-(((1-methyl-1H-pyrazol-3-yl)amino)methyl)phenyl)-1,2,5-thiadiazolidin-3-one 1,1-dioxide;
5-(4-(((1,3,4-thiadiazol-2-yl)amino)methyl)-2-fluoro-6-hydroxyphenyl)-1,2,5-thiadiazolidin-3-one 1,1-dioxide;
5-(2-fluoro-6-hydroxy-4-(((5-methyl-1H-imidazol-2-yl)amino)methyl)phenyl)-1,2,5-thiadiazolidin-3-one 1,1-dioxide;
5-(2-fluoro-6-hydroxy-4-((thiazol-2-ylamino)methyl)phenyl)-1,2,5-thiadiazolidin-3-one 1,1-dioxide;
5-(4-(((1H-imidazol-2-yl)amino)methyl)-2-fluoro-6-hydroxyphenyl)-1,2,5-thiadiazolidin-3-one 1,1-dioxide;
5-(2-fluoro-6-hydroxy-4-(((1-methyl-1H-imidazol-2-yl)amino)methyl)phenyl)-1,2,5-thiadiazolidin-3-one 1,1-dioxide;
5-(2-fluoro-6-hydroxy-4-(((5-methyl-1,3,4-thiadiazol-2-yl)amino)methyl)phenyl)-1,2,5-thiadiazolidin-3-one 1,1-dioxide;
5-(2-fluoro-6-hydroxy-4-(((4-methylthiazol-2-yl)amino)methyl)phenyl)-1,2,5-thiadiazolidin-3-one 1,1-dioxide;
5-(2-fluoro-6-hydroxy-4-(((3-methylisothiazol-5-yl)amino)methyl)phenyl)-1,2,5-thiadiazolidin -3-one 1,1-dioxide;
5-(2-fluoro-6-hydroxy-4-(((3-methyl-1H-pyrazol-5-yl)amino)methyl)phenyl)-1,2,5-thiadiazolidin-3-one 1,1-dioxide;
or pharmaceutically acceptable salts thereof.

In one embodiment, the invention comprises a pharmaceutical composition comprising a compound of Formula (I), a pharmaceutically acceptable salt thereof, and at least one pharmaceutically acceptable carrier.

In another embodiment, the invention comprises a method for treating cancer comprising administering to said patient a therapeutically effective amount of a compound of Formula (I) or a pharmaceutically acceptable salt thereof wherein the cancer/disease is selected from: human cancers, carcinomas, sarcomas, adenocarcinomas, papillary adenocarcinomas, lymphomas, leukemias, melanomas, solid lymphoid cancers, kidney cancer, breast cancer, lung cancer, bladder cancer, colon cancer, ovarian cancer, prostate cancer, pancreatic cancer, stomach cancer, brain cancer, head and neck cancer, skin cancer, uterine, testicular, glioma, esophagus, liver cancer, including hepatocarcinoma, lymphoma, including B-acute lymphoblastic lymphoma, non-Hodgkin's lymphomas, Burkitt's lymphoma, Small lymphomas, Hodgkin's lymphoma, leukemia, and multiple myeloma.

In another embodiment, the invention comprises a method of treating cancer in a patient in need thereof, comprising administering to the patient an effective amount of a compound of formula I in combination with an additional therapeutic agent.

In one embodiment, the additional therapeutic agent is an immunotherapeutic agent.

In another embodiment, the immunotherapeutic agent is selected from the group consisting of an anti-PD-1 antibody, an anti-PD-L1 antibody, and an anti-CTLA-4 antibody.

In one embodiment, the method of treating cancer in a patient in need thereof, comprises administering to the patient an effective amount of a pharmaceutically acceptable composition of the compound of formula I.

In another embodiment, the method of treating cancer is selected from radiation, surgery, chemotherapy, or administration of a biologic drug.

In one embodiment, the method of treating cancer is the administration of a biologic drug and the biologic drug is a drug that stimulates the immune system.

In another embodiment, the method of treating cancer comprises administering to the subject an inhibitor of DGKα and/or DGKζ, an antagonist of the PD1/PD-L1 axis and an antagonist of CTLA4.

These embodiments are not intended to limit the scope of the invention.

### Synthetic Methods

The compounds of the invention may be prepared by the methods and examples presented below and by methods known to those of ordinary skill in the art. In each of the examples below, the R groups are as defined above for each formula unless noted. Optimum reaction conditions and reaction times may vary according to the reactants used. Unless otherwise specified, solvents, temperatures, pressures, and other reaction conditions may be readily selected by one of ordinary skill in the art.

The intermediates used in the syntheses below are either commercially available or easily prepared by methods known to those skilled in the art. Reaction progress may be monitored by conventional methods such as thin-layer chromatography (TLC) or high-pressure liquid chromatography-mass spec (HPLC-MS). Intermediates and products may be purified by methods known in the art, including column chromatography, HPLC, preparative TLC or Preparatory HPLC.

### Preparation of Synthetic Key Intermediates (Int-2)

### Preparation of 5-(4-bromo-2-fluoro-6-((4-methoxybenzyl)oxy)phenyl)-1,2,5-thiadiazolidin -3-one 1,1-dioxide (Int-2) as shown in Scheme 1

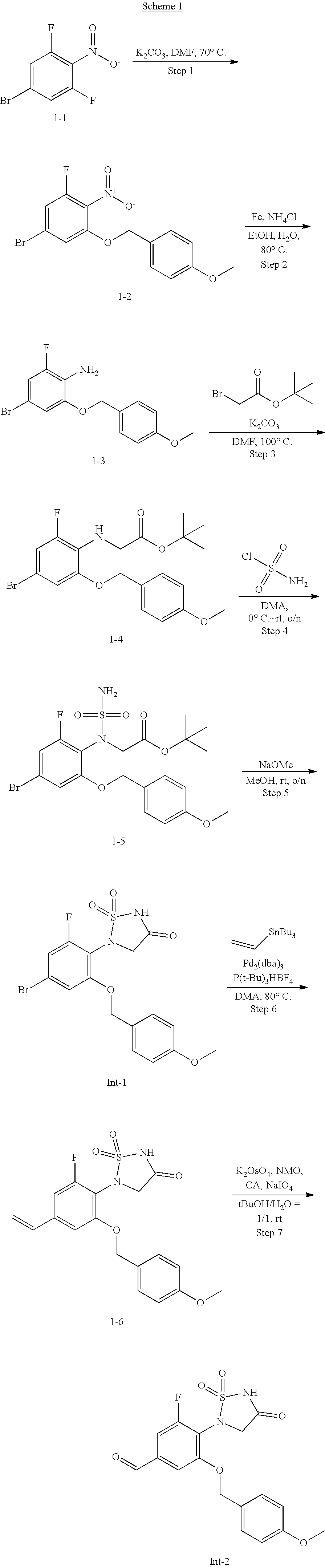

### Step 1: Synthesis of 5-bromo-1-fluoro-3-((4-methoxybenzyl)oxy)-2-nitrobenzene (1-2)

To a stirred solution of 5-bromo-1,3-difluoro-2-nitro-benzene (10 g, 42.02 mmol) and (4-methoxyphenyl)methanol (6.1 g, 44.12 mmol) in DMF (100 mL) was added K₂CO₃ (17.4 g, 126.06 mmol) in portions at room temperature. The resulting mixture was stirred over night at 70° C. under a nitrogen atmosphere. TLC showed the reaction was completed. The reaction mixture was diluted with water (300 mL) and extracted with ethyl acetate (3*300 mL). The combined organic layers were washed with brine and dried over anhydrous sodium sulfate. After filtration, the filtrate was concentrated under reduced pressure. The resulting residue was purified by a silica gel column chromatography (ethyl acetate/petroleum ether= 1/20) to afford the desired product 5-bromo-1-fluoro-3-[(4-methoxyphenyl)methoxy]-2-nitro-benzene (10 g, 66.8% yield) as a light yellow solid.

### Step 2: Synthesis of 4-bromo-2-fluoro-6-((4-methoxybenzyl)oxy)aniline (1-3)

To a stirred solution of 5-bromo-1-fluoro-3-[(4-methoxyphenyl)methoxy]-2-nitro-benzene (10 g, 28.08 mmol) in ethanol (200 mL) and water (20 mL) were added NH4Cl (15.16 g, 280.79 mmol), and Fe (15.68 g, 280.79 mmol) at room temperature. The resulting mixture was stirred at 80° C. for overnight under a nitrogen atmosphere. LCMS showed the reaction was completed. The reaction mixture was filtrated. The filtrate was concentrated under reduced pressure. The resulting residue was purified by column chromatography on a silica gel (PE/EA=9/1) to afford the desired product 4-bromo-2-fluoro-6-[(4-methoxyphenyl)methoxy ]aniline (6 g, 65.50% yield) as a light yellow solid. MS: m/z: Calc'd for C₁₄H₁₃BrFNO₂ [M+H]⁺326, found 326.

### Step 3: Synthesis of tert-butyl (4-bromo-2-fluoro-6-((4-methoxybenzyl)oxy)phenyl)glycinate (1-4)

To a stirred solution of 4-bromo-2-fluoro-6-[(4-methoxyphenyl)methoxy ]aniline (5.9 g, 18.09 mmol) and tert-butyl 2-bromoacetate (10.58 g, 54.27 mmol) in DMF (90 mL) was added K₂CO₃ (7.49 g, 54.27 mmol) at room temperature. The resulting mixture was stirred at 100° C. for 48 h. LCMS showed the starting material was consumed completely. The reaction mixture was filtered, and the filtrate was washed with brine for 3 times. The organic phase was dried over sodium sulfate, filtrated, and concentrated. The residue was subjected to silica gel column chromatography to obtain the product as a mixture. The mixture was further purified by reversed-phase flash chromatography (0.05% NH₄HCO₃ in H₂O/ACN) to afford tert-butyl 2-[4-bromo-2-fluoro-6-[(4-methoxyphenyl)methoxy]anilino]acetate (5 g, 62.70% yield) as a white solid. MS: m/z: Calc'd for C₂₀H₂₃BrFNO₄ [M+H]⁺440, found 440.

### Step 4: Synthesis of tert-butyl N-(4-bromo-2-fluoro-6-((4-methoxybenzyl)oxy)phenyl)-N -sulfamoylglycinate (1-5)

To a stirred solution of tert-butyl 2-[4-bromo-2-fluoro-6-[(4-methoxyphenyl)methoxy]anilino]acetate (3.3 g, 7.49 mmol) in DMA (80 mL) was added a solution of sulfamoyl chloride (2.6 g, 22.48 mmol) in DMA (4 mL) at 0° C. The reaction mixture was stirred at room temperature for overnight. LCMS showed the starting material was consumed completely. The mixture was diluted with ethyl acetate (300 mL) and washed with brine for 6 times until the DMA was washed out completely. The organic phase was dried over anhydrous sodium sulfate, filtrated and concentrated to obtain tert-butyl 2-[4-bromo-2-fluoro-6-[(4-methoxyphenyl)methoxy]-N-sulfamoyl-anilino]acetate (4 g, 7.70 mmol, 102.70% yield) as a brown oil. MS: m/z: Calc'd for C₂₀H₂₄BrFN₂O₆S [M−H]⁻517, found 517.

### Step 5: Synthesis of 5-(4-bromo-2-fluoro-6-((4-methoxybenzyl)oxy)phenyl)-1,2,5-thiadiazolidin-3-one 1,1-dioxide (Int-1)

To a stirred solution of tert-butyl 2-[4-bromo-2-fluoro-6-[(4-methoxyphenyl)methoxy]-N -sulfamoyl-anilino]acetate (4 g, 7.70 mmol) in Methanol (20 mL) was added 30% NaOMe in MeOH (8.32 g, 46.30 mmol) at 0° C. The mixture was stirred at room temperature for overnight. LCMS showed the starting material was consumed completely. The mixture was concentrated. The resulting suspension was dissolved with water (200 mL) and extracted with ethyl acetate. The organic phase was separated and discarded. The aqueous layer was diluted with ethyl acetate and acidified by 1N HCl solution to pH=3. and extracted with ethyl acetate for 3 times. The combined organic layers were washed with brine, dried over sodium sulfate, filtered, and concentrated in vacuum. The resulting residue was further purified by reversed-phase column (0.05% NH₄CO₃ in H₂O and MeCN) to afford 5-[4-bromo-2-fluoro-6-[(4-methoxyphenyl)methoxy]phenyl]-1,1-dioxo-1,2,5-thiadiazolidin-3-one (2.50 g, 5.61 mmol, 72.90% yield) as an off-white solid. MS: m/z: Calc'd for C₁₆H₁₄BrFN₂O₅S [M−H]⁻443, found 443.

### Step 6: Synthesis of 5-(2-fluoro-6-((4-methoxybenzyl)oxy)-4-vinylphenyl)-1,2,5-thiadiazolidin-3-one 1,1-dioxide (1-6)

To a solution of 5-[4-bromo-2-fluoro-6-[(4-methoxyphenyl)methoxy]phenyl]-1,1-dioxo-1,2,5-thiadiazolidin-3-one (2 g, 4.49 mmol) and tributyl(vinyl)stannane (2.85 g, 8.98 mmol) in DMA (20 mL) were added P(t-Bu)₃HBF₄ (0.43 g, 0.90 mmol) and Pd₂(dba)₃ (0.41 g, 0.45 mmol). The resulting mixture was purged with nitrogen for 5 minutes. Then, the mixture was stirred at 80° C. for 12 h. LCMS showed the starting material was consumed completely. The reaction mixture was filtrated, and the filtrate was directly purified by a reversed-phase column to obtain 5-[2-fluoro-6-[(4-methoxyphenyl)methoxy]-4-vinyl-phenyl]-1,1-dioxo-1,2,5-thiadiazolidin-3-one (1.2 g, 3.05 mmol, 68.08% yield) as a light yellow semi-solid. MS: m/z: Calc'd for C₁₈H₁₈FN₂O₅S [M−H]⁻391, found 391.

### Step 7: Synthesis of 4-(1,1-dioxido-4-oxo-1,2,5-thiadiazolidin-2-yl)-3-fluoro-5-((4-methoxybenzyl)oxy)benzaldehyde (Int-2)

To a stirred solution of 5-[2-fluoro-6-[(4-methoxyphenyl)methoxy]-4-vinyl-phenyl]-1,1-dioxo -1,2,5-thiadiazolidin-3-one (970 mg, 2.47 mmol), Citric acid (1.04 g, 4.94 mmol) and NMO (579.18 mg, 4.94 mmol) in tert-butanol (6 mL) and Water (6 mL) was added K₂OsO₄ (91.07 mg, 0.25 mmol). The resulting mixture was stirred at room temperature for 1 h. LCMS showed the starting material was converted to the intermediate completely. Then, NaIO₄ (1.07 mL, 7.42 mmol) was added to the mixture at 0° C. The resulting mixture was stirred at room temperature for 2 h. LCMS showed the reaction was completed. The reaction mixture was diluted with water and extracted with ethyl acetate for 4 times. The organic phase was dried over sodium sulfate, filtrated, and concentrated. The resulting residue was purified by reversed-phase column (0.05% NH₄CO₃, H₂O/ACN) to obtain 3-fluoro-5-[(4-methoxyphenyl)methoxy]-4-(1,1,4-trioxo-1,2,5-thiadiazolidin-2-yl)benzaldehyde (500 mg, 1.26 mmol, 51.20% yield) as a brown solid. MS: m/z: Calc'd for C₁₇H₁₅FN₂O₆S [M−H]⁻393, found 393.

### PREPARATION OF EXAMPLES

### Example 1: 5-[4-[[[4-(2,4-dimethylphenyl)thiazol-2-yl]-methyl-amino]methyl]-2-fluoro-6-hydroxy-phenyl]-1,1-dioxo-1,2,5-thiadiazolidin-3-one

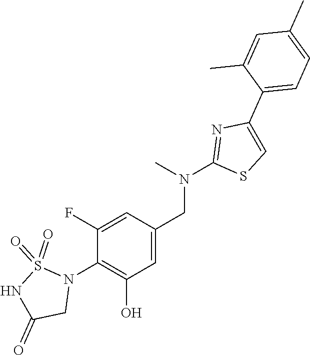 [SMILES: Cc1ccc(-c2csc(N(C)Cc3cc(O)c(N4CC(=O)NS4(=O)=O)c(F)c3)n2)c(C)c1]

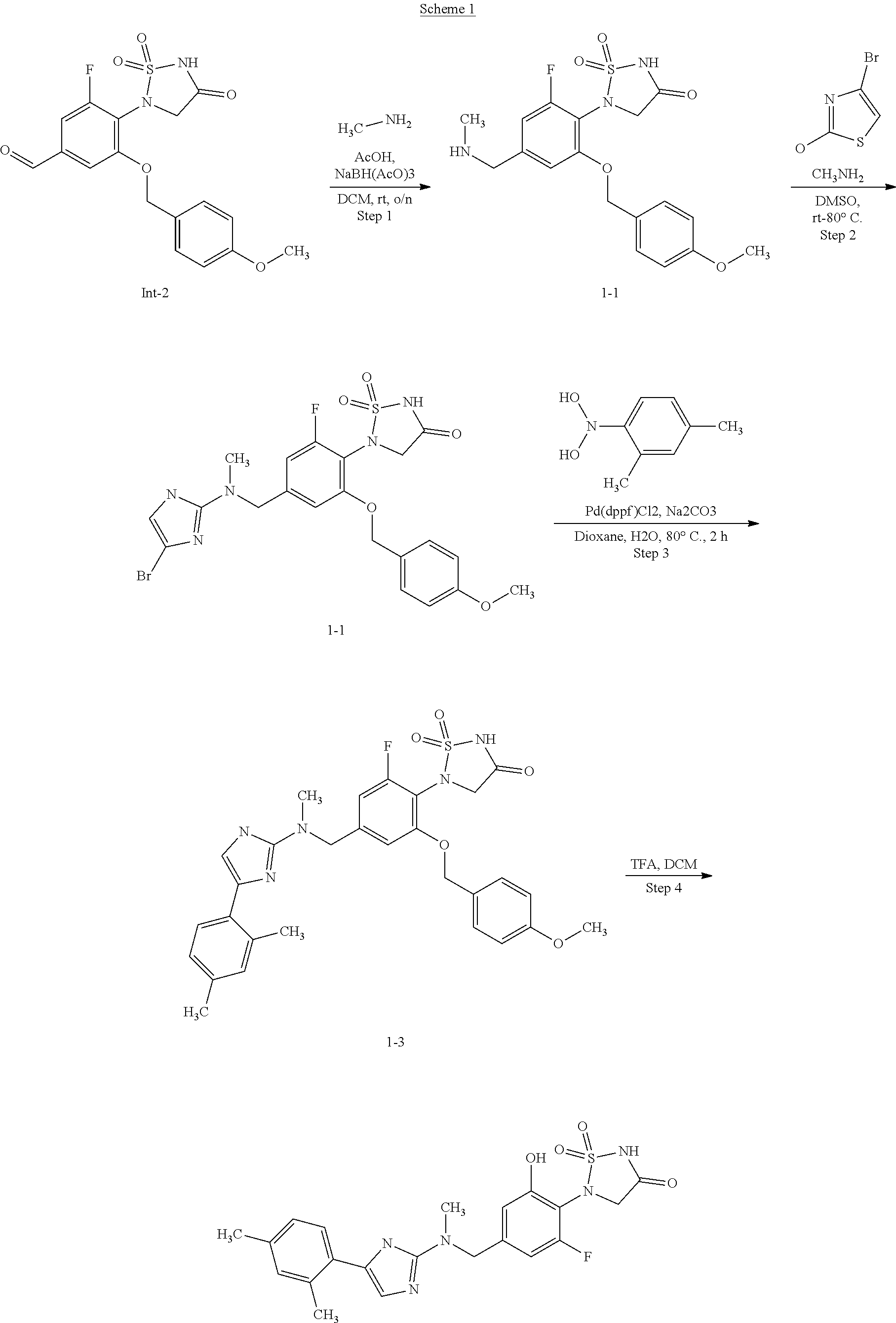

Step 1: To a stirred solution of methanamine (70.88 mg, 2.28 mmol) and 3-fluoro-5-[(4-methoxyphenyl)methoxy]-4-(1,1,4-trioxo-1,2,5-thiadiazolidin-2-yl)benzaldehyde (Int-2, 300 mg, 0.76 mmol) in methanol (10 mL) were added acetic acid (91.36 mg, 1.52 mmol). The mixture was stirred at room temperature for 1 h. Then, NaBH₃CN (191.09 mg, 3.04 mmol) was added to the reaction mixture at 0° C. The resulting mixture was stirred at room temperature for additional 1 h. After completion of the reaction monitored by LCMS, the mixture was concentrated. The resulting residue was purified by a reversed-phase column to obtain 5-[2-fluoro-6-[(4-methoxyphenyl)methoxy]-4-(methylaminomethyl)phenyl]-1,1-dioxo-1,2,5-thiadiazolidin-3-one (210 mg, 0.51 mmol, 67.42% yield) as a white solid. MS: m/z: Calc'd for C₁₈H₂₀FN₃O₅S [M+H]⁺410, found 410.

Step 2: To a stirred solution of 5-[2-fluoro-6-[(4-methoxyphenyl)methoxy]-4-(methylaminomethyl)phenyl]-1,1-dioxo-1,2,5-thiadiazolidin-3-one (205 mg, 0.50 mmol) and 4-bromo-2-chloro-thiazole (198.75 mg, 1 mmol) in DMSO (4 mL) was added DIEA (0.02 mL, 1.5 mmol). The mixture solution was stirred at 80° ° C.for 16 h. LCMS showed the starting material was consumed completely. The mixture was purified by a reversed-phase column to obtain 5-[4-[[(4-bromothiazol-2-yl)-methyl-amino]methyl]-2-fluoro-6-[(4-methoxyphenyl)methoxy]phenyl]-1,1-dioxo-1,2,5-thiadiazolidin-3-one (120 mg, 0.21 mmol, 41.94% yield) as a brown solid. MS: m/z: Calc'd for C₂₁H₂₀BrFN₄O₅S₂ [M+H]⁺571, found 571.

Step 3: To a stirred solution of 5-[4-[[(4-bromothiazol-2-yl)-methyl-amino]methyl]-2-fluoro-6-[(4-methoxyphenyl)methoxy]phenyl]-1,1-dioxo-1,2,5-thiadiazolidin-3-one (90 mg, 0.16 mmol), (2,4-dimethylphenyl)boronic acid (35.43 mg, 0.24 mmol) and Na₂CO₃ (50.08 mg, 0.47 mmol) in 1,4-dioxane (10 mL) and water (1 mL) was added Pd(dppf)Cl₂ (38.59 mg, 0.05 mmol) under nitrogen. The resulting mixture was stirred at 80° C. for 2 h. After completion of the reaction monitored by LCMS, the mixture was concentrated. The resulting residue was purified by a reversed-phase column to obtain 5-[4-[[[4-(2,4-dimethylphenyl)thiazol-2-yl]-methyl -amino]methyl]-2-fluoro-6-[(4-methoxyphenyl)methoxy]phenyl]-1,1-dioxo-1,2,5-thiadiazolidin -3-one (60 mg, 0.10 mmol, 63.84% yield) as a yellow solid. MS: m/z: Calc'd for C₂₉H₂₉FN₄O₅S₂ [M+H]⁺597, found 597.

Step 4: To a stirred solution of 5-[4-[[[4-(2,4-dimethylphenyl)thiazol-2-yl]-methyl -amino]methyl]-2-fluoro-6-[(4-methoxyphenyl)methoxy]phenyl]-1,1-dioxo-1,2,5-thiadiazolidin -3-one (60 mg, 0.10 mmol) in DCM (3 mL) was added TFA (3 mL), and the mixture was stirred at room temperature for 2 h. Upon completion, the reaction mixture was concentrated. The resulting residue was purified by reversed-flash (0.05% NH₄HCO₃ in H₂O and ACN) and further purified by Prep-HPLC to obtain 5-[4-[[[6-(dimethylamino)-3-pyridyl]amino]methyl]-2-fluoro-6-hydroxy-phenyl]-1,1-dioxo-1,2,5-thiadiazolidin-3-one (3 mg, 5.69% yield) as a green solid. MS: m/z: Calc'd for C₂₁H₂₁FN₄O₄S₂, [M+H]⁺477; Found 477. ¹H NMR (400 MHZ, DMSO-d₆) δ 10.36 (s, 1H), 7.47 (d, J=7.8 Hz, 1H), 7.06-6.95 (m, 2H), 6.79 (s, 1H), 6.72-6.64 (m, 2H), 4.67 (s, 2H), 4.31 (s, 2H), 3.09 (s, 3H), 2.36 (s, 3H), 2.28 (s, 3H).

Prep-HPLC purification conditions: Column: Welch Utimate AQ C18, 50*250 mm, 10 μm; Mobile Phase A: Water (0.1% TFA), Mobile Phase B: ACN; Flow rate: 100 mL/min; Gradient: 40% B to 65% B in 20 min, 65% B; Wavelength: 254 nm.

### Example 2: 5-[2-fluoro-6-hydroxy-4-[(1H-pyrazol-3-ylamino)methyl]phenyl]-1,1-dioxo -1,2,5-thiadiazolidin-3-one

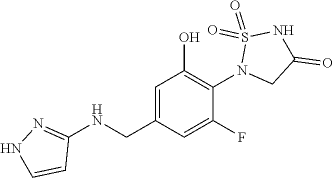 [SMILES: O=C1CN(c2c(O)cc(CNc3cc[nH]n3)cc2F)S(=O)(=O)N1]

The title compound was prepared in 8.78% overall yield as a white solid according to the preparation of EXAMPLE 4 using 1H-pyrazol-3-amine in STEP 1. MS: m/z: Calc'd for C₁₂H₁₂FN₅O₄S, [M+H]⁺342; Found 342. ¹H NMR (400 MHZ, DMSO-d₆) δ 9.39 (s, 1H), 7.65 (s, 1H), 6.60-6.50 (m, 3H), 3.96 (s, 2H), 3.62 (s, 2H).

Prep-HPLC purification conditions: SunFire Prep C18 OBD Column, 19*150 mm, 5 μm; Mobile Phase A: Water (0.05% TFA), Mobile Phase B: ACN; Flow rate: 60 mL/min; Gradient: 20% B to 40% B in 6.5 min, 40% B; Wavelength: 254/210 nm.

### Example 3: 5-[2-fluoro-6-hydroxy-4-[(isoxazol-3-ylamino)methyl]phenyl]-1,1-dioxo-1,2,5-thiadiazolidin-3-one

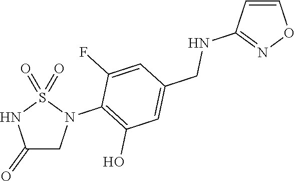 [SMILES: O=C1CN(c2c(O)cc(CNc3ccon3)cc2F)S(=O)(=O)N1]

The title compound was prepared in 4.75% overall yield as a white solid according to the preparation of EXAMPLE 4 using isoxazol-3-amine in STEP 1. MS: m/z: Calc'd for C₁₂H₁₁FN₄O₅S, [M+H]⁺343; Found 343. ¹H NMR (400 MHZ, DMSO-d₆) δ 8.35 (d, J=1.8 Hz, 1H), 6.71 (d, J=1.9 Hz, 1H), 6.66 (dd, J=10.7, 1.8 Hz, 1H), 6.00 (d, J=1.8 Hz, 1H), 4.20 (d, J=10.8 Hz, 4H).

Prep-HPLC purification conditions: Column: SunFire Prep C18 OBD Column, 19*150 mm, 5 μm; Mobile Phase A: Water (0.1% FA), Mobile Phase B: ACN; Flow rate: 60 mL/min; Gradient: 40% B to 60% B in 6.5 min, 60% B; Wavelength: 254/210 nm.

### Example 4: 5-[2-fluoro-6-hydroxy-4-[[(5-methylisoxazol-3-yl) amino]methyl]phenyl]-1,1-dioxo-1,2,5-thiadiazolidin-3-one

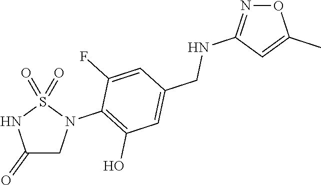 [SMILES: Cc1cc(NCc2cc(O)c(N3CC(=O)NS3(=O)=O)c(F)c2)no1]

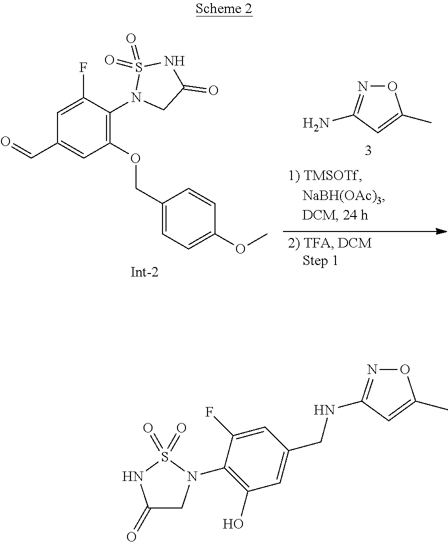

To a stirred solution of 3-fluoro-5-[(4-methoxyphenyl)methoxy]-4-(1,1,4-trioxo-1,2,5-thiadiazolidin-2-yl)benzaldehyde (100 mg, 0.25 mmol) and 5-methylisoxazol-3-amine (61.66 mg, 0.38 mmol) in DCM (8 mL) was added TMSOTf (84.44 mg, 0.38 mmol) at 0° C. The reaction mixture was stirred at room temperature for 2 h. The mixture was cooled to 0° C., and NaBH(AcO)₃ (107.51 mg, 0.51 mmol) was added slowly to the above mixture. After the addition, the resulting mixture was stirred at room temperature for additional 16 h. LCMS showed the starting material was consumed completely (approximate 50% of desired product, was observed together with 10% of PMB protected intermediate). TFA (10 mL) was added to the reaction mixture at 0° C. The resulting mixture was stirred at room temperature for another 3 h. LCMS showed PMB protecting group was totally cleaved, and the reaction mixture was concentrated. The residue was purified by a reversed-phase column chromatography (0.05% NH₄HCO₃ in H₂O and MeCN) and further purified by Prep-HPLC to obtain 5-[2-fluoro-6-hydroxy-4-[[(5-methylisoxazol-3-yl)amino]methyl]phenyl]-1,1-dioxo-1,2,5-thiadiazolidin-3-one (9 mg, 8.02% yield) as a yellow solid. MS: m/z: Calc'd for C₁₃H₁₃FN₄O₅S, [M+H]⁺357; Found 357. ¹H NMR (400 MHZ, DMSO-d₆) δ 7.60 (s, 2H), 6.65 (s, 1H), 6.64-6.52 (m, 2H), 5.66 (s, 1H), 4.13 (d, J=6.2 Hz, 2H), 3.94 (d, J=2.2 Hz, 2H), 2.21 (s, 3H).

Prep-HPLC purification conditions: Column: XBridge Prep Phenyl OBD Column, 19*100 mm, 5 μm; Mobile Phase A: Water (10 mmol/L NH₄HCO₃), Mobile Phase B: ACN; Flow rate: 50 mL/min; Gradient: 35% B to 45% B in 8 min, 45% B; Wavelength: 254/210 nm.

### Example 5: 5-[2-fluoro-6-hydroxy-4-[(1H-1,2,4-triazol-3-ylamino)methyl]phenyl]-1,1-dioxo -1,2,5-thiadiazolidin-3-one

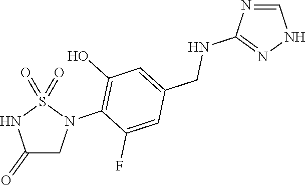 [SMILES: O=C1CN(c2c(O)cc(CNc3nc[nH]n3)cc2F)S(=O)(=O)N1]

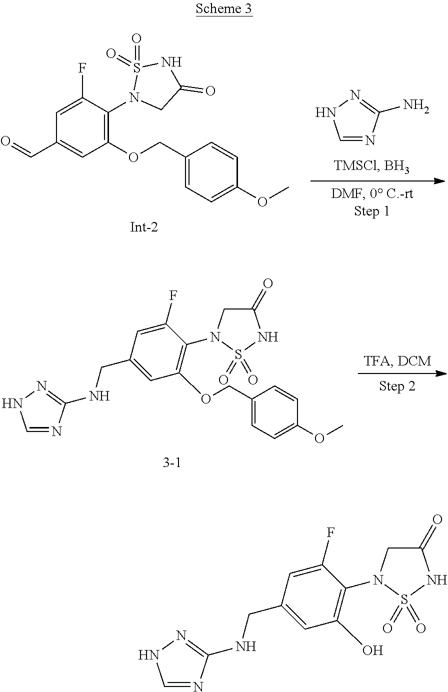 [SMILES: COc1ccc(COc2cc(C=O)cc(F)c2N2CC(=O)NS2(=O)=O)cc1 ; Nc1nc[nH]n1 ; COc1ccc(COc2cc(CNc3nc[nH]n3)cc(F)c2N2CC(=O)NS2(=O)=O)cc1 ; O=C1CN(c2c(O)cc(CNc3nc[nH]n3)cc2F)S(=O)(=O)N1]

Step 1: To a stirred solution of 3-fluoro-5-[(4-methoxyphenyl) methoxy]-4-(1, 1,4-trioxo-1,2,5-thiadiazolidin-2-yl) benzaldehyde (Int-2, 100 mg, 0.25 mmol) and 1H-1,2,4-triazol-3-amine (34.2 mg, 0.28 mmol) in dry DMF (6 mL) was added TMSCI (0.08 mL, 0.62 mmol) dropwise at 0° C., and the resulting mixture was stirred at room temperature for 30 mins. The reaction mixture was then cooled to 60° C. and a solution of BH₃ in THF (1 M, 0.45 mL, 0.46 mmol) was added slowly with a syringe. After the addition, the reaction mixture was stirred at room temperature for 1 h. LCMS showed the reaction was completed. The resulting solution was quenched with ice water (1 ml) and directly purified by reversed-phase column (0.05% NH₄HCO₃ in H₂O and MeCN) to obtain 5-[2-fluoro-6-hydroxy-4-[(1H-1,2,4-triazol-3-ylamino)methyl]phenyl]-1,1-dioxo-1,2,5-thiadiazolidin-3-one 5-(4-(((2,6-dimethylpyridin-4-yl)amino)methyl)-2-fluoro-6-((4-methoxybenzyl)oxy)phenyl)-1,2,5-thiadiazolidin-3-one 1,1-dioxide (76 mg, 0.14 mmol, 56.40% yield) as a yellow solid. MS: m/z: Calc'd for C₂₄H₂₀ClFN₄O₅S [M+H]⁺531; Found, 531.

Step 2: The title compound was prepared in 5.99% yield as a white solid according to the preparation of EXAMPLE 1 using 3-1 in STEP 2.

MS: m/z: Calc'd for C₁₁H₁₁FN₆O₄S, [M+H]⁺343; Found 343. ¹H NMR (400 MHZ, DMSO -d₆+D₂O) δ 6.86-6.50 (m, 2H), 4.23 (s, 2H), 3.94 (s, 2H).

Prep-HPLC purification conditions: Column: XBridge BEH C18 OBD Prep Column, 19*250 mm, 5 μm; Mobile Phase A: Water (10 mmol/L NH₄HCO₃), Mobile Phase B: ACN; Flow rate: 25 mL/min; Gradient: 8% B to 20% B in 6 min, 20% B; Wavelength: 254 nm.

### Example 6: 5-[2-fluoro-6-hydroxy-4-[[(1-methylpyrazol-3-yl)amino]methyl]phenyl]-1,1-dioxo-1,2,5-thiadiazolidin-3-one

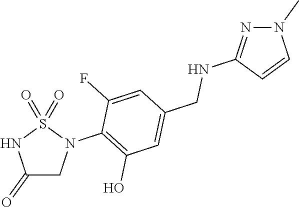 [SMILES: Cn1ccc(NCc2cc(O)c(N3CC(=O)NS3(=O)=O)c(F)c2)n1]

The title compound was prepared in 6.93% overall yield as a white solid according to the preparation of EXAMPLE 4 using 1-methylpyrazol-3-amine in STEP 1. MS: m/z: Calc'd for C₁₃H₁₄FN₅O₄S, [M+H]⁺356; Found 356. ¹H NMR (400 MHZ, DMSO-d₆+D₂O) δ 7.52-7.51 (m, 1H), 6.72-6.59 (m, 2H), 4.17 (s, 2H), 4.09 (s, 2H), 3.62 (d, J=1.6 Hz, 3H).

Prep-HPLC purification conditions: Column: SunFire Prep C18 OBD Column, 19*150 mm, 5 μm; Mobile Phase A: Water (0.1% FA), Mobile Phase B: ACN; Flow rate: 25 mL/min; Gradient: 15% B to 28% B in 5.5 min, 28% B; Wavelength: 210/254 nm.

### Example 7: 5-[2-fluoro-6-hydroxy-4-[(1,3,4-thiadiazol-2-ylamino)methyl]phenyl]-1,1-dioxo-1,2,5-thiadiazolidin-3-one

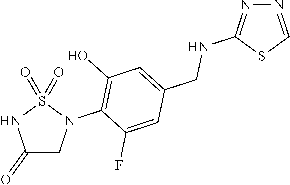 [SMILES: O=C1CN(c2c(O)cc(CNc3nncs3)cc2F)S(=O)(=O)N1]

The title compound was prepared in 8.99% overall yield as a white solid according to the preparation of EXAMPLE 5 using 1,3,4-thiadiazol-2-amine in STEP 1. MS: m/z: Calc'd for C₁₁H₁₀FN₅O₄S₂, [M+H]⁺360; Found 360. ¹H NMR (400 MHz, DMSO-d₆) δ 8.64 (s, 1H), 8.27 (t, J=5.8 Hz, 1H), 6.87-6.52 (m, 2H), 4.39 (d, J=5.8 Hz, 2H), 3.93 (s, 2H).

Prep-HPLC purification conditions: Column: XBridge BEH C18 OBD Prep Column, 19*250 mm, 5 μm; Mobile Phase A: Water (10 mmol/L NH₄HCO₃), Mobile Phase B: ACN; Flow rate: 25 mL/min; Gradient: 10% B to 25% B in 7 min, 25% B; Wavelength: 254 nm.

### Example 8: 5-[2-fluoro-6-hydroxy-4-[[(5-methyl-1H-imidazol-2-yl)amino]methyl]phenyl]-1,1-dioxo-1,2,5-thiadiazolidin-3-one

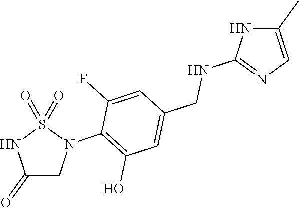 [SMILES: Cc1cnc(NCc2cc(O)c(N3CC(=O)NS3(=O)=O)c(F)c2)[nH]1]

The title compound was prepared in 7.52% overall yield as a white solid according to the preparation of EXAMPLE 5 using 5-methyl-1H-imidazol-2-amine; hydrochloride in STEP 1. MS: m/z: Calc'd for C₁₃H₁₄FN₅O4S, [M+H]⁺356; Found 356. ¹H NMR (400 MHZ, DMSO-d₆) δ 11.75 (s, 1H), 9.45 (s, 1H), 7.20 (s, 2H), 6.56 (d, J=9.5 Hz, 2H), 3.99 (d, J=25.2 Hz, 2H), 3.70 (s, 2H), 2.11 (s, 3H).

Prep-HPLC purification conditions: Column: SunFire Prep C18 OBD Column, 19*150 mm, 5 μm; Mobile Phase A: Water (0.05%TFA), Mobile Phase B: ACN; Flow rate: 60 mL/min; Gradient: 22% B to 40% B in 7 min, 45% B; Wavelength: 254/210 nm.

### Example 9: 5-[2-fluoro-6-hydroxy-4-[(thiazol-2-ylamino)methyl]phenyl]-1,1-dioxo-1,2,5-thiadiazolidin-3-one

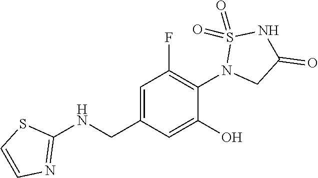 [SMILES: O=C1CN(c2c(O)cc(CNc3nccs3)cc2F)S(=O)(=O)N1]

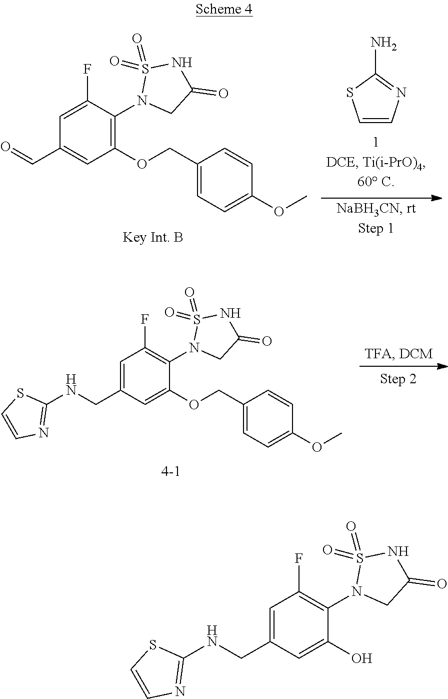 [SMILES: COc1ccc(COc2cc(C=O)cc(F)c2N2CC(=O)NS2(=O)=O)cc1 ; Nc1nccs1 ; COc1ccc(COc2cc(CNc3nccs3)cc(F)c2N2CC(=O)NS2(=O)=O)cc1 ; O=C1CN(c2c(O)cc(CNc3nccs3)cc2F)S(=O)(=O)N1]

Step 1: To a stirred solution of 3-fluoro-5-[(4-methoxyphenyl)methoxy]-4-(1,1,4-trioxo-1,2,5-thiadiazolidin-2-yl)benzaldehyde (60 mg, 0.15 mmol) and thiazol-2-amine (36 mg, 0.36 mmol) in DCE (10 mL) added Ti(i-PrO)₄ (172.83 mg, 0.61 mmol) at 0° C. The resulting mixture was stirred at 60° C. for 12 h. NaBH₃CN (19.47, 0.30 mmol) was added to the mixture at 0° C. The resulting mixture was stirred at room temperature for additional 2 h. Upon completion, the reaction mixture was concentrated. The resulting residue was dissolved with DMSO and directly purified by a reversed-phase column (0.05% TFA in H₂O and MeCN) to obtain 5-[2-fluoro-6-[(4-methoxyphenyl)methoxy]-4-[(thiazol-2-ylamino)methyl]phenyl]-1,1-dioxo-1,2,5-thiadiazolidin-3-one (70.0 mg, 0.14 mmol, 96.15% yield) as a white solid. MS: m/z: Calc'd for C₂₀H₁₉FN₄O₅S₂ [M+H]⁺479; Found 479.

Step 2: To a stirred solution of 5-[2-fluoro-6-[(4-methoxyphenyl)methoxy]-4-[(thiazol-2-ylamino)methyl]phenyl]-1,1-dioxo-1,2,5-thiadiazolidin-3-one (70 mg, 0.15 mmol) in DCM (6 mL) was added TFA (2 mL) at room temperature. The resulting mixture was stirred at room temperature for 12 h. Upon completion, the reaction mixture was concentrated. The resulting residue was purified by a reversed-phase column chromatography (0.05% TFA in H₂O and MeCN) and further purified by Prep-HPLC to afford 5-[2-fluoro-6-hydroxy-4-[(thiazol-2-ylamino)methyl]phenyl]-1,1-dioxo-1,2,5-thiadiazolidin-3-one (5.7 mg, 0.01 mmol, 10.64% yield) as a brown solid. MS: m/z: Calc'd for C₁₂H₁₁FN₄O₄S₂ [M+H]⁺359, found 359. ¹H NMR (400 MHZ, DMSO-d₆) δ 7.13 (t, J=3.6 Hz, 1H), 6.79-6.74 (m, 1H), 6.66 (d, J=12.0 Hz, 2H), 4.40 (s, 2H), 4.01 (d, J=2.2 Hz, 2H).

Prep-HPLC purification conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 5% B to 35% B in 8 min, 35% B; Wavelength: 254/210 nm.

### Example 10: 5-[2-fluoro-6-hydroxy-4-[(1H-imidazol-2-ylamino)methyl]phenyl]-1,1-dioxo -1,2,5-thiadiazolidin-3-one

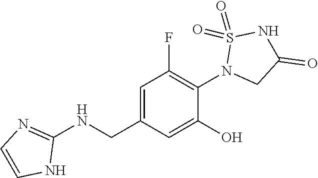 [SMILES: O=C1CN(c2c(O)cc(CNc3ncc[nH]3)cc2F)S(=O)(=O)N1]

The title compound was prepared in 8.56% overall yield as a white solid according to the preparation of EXAMPLE 5 using 1H-imidazol-2-amine in STEP 1. MS: m/z: Calc'd for C₁₂H₁₂FN₅O₄S, [M+H]⁺342; Found 342. 1H NMR (400 MHZ, DMSO-d₆+D₂O) δ 6.86 (s, 2H), 6.70 (d, J=8.4 Hz, 2H), 4.38 (s, 2H), 4.06 (s, 2H).

Prep-HPLC purification conditions: Column: SunFire Prep C18 OBD Column, 19*150 mm, 5 μm; Mobile Phase A: Water (0.1% FA), Mobile Phase B: ACN; Flow rate: 60 mL/min; Gradient: 10% B to 30% B in 6.59 min, 30% B; Wavelength: 254/210 nm.

### Example 11: 5-[2-fluoro-6-hydroxy-4-[[(1-methylimidazol-2-yl)amino]methyl]phenyl]-1,1-dioxo-1,2,5-thiadiazolidin-3-one

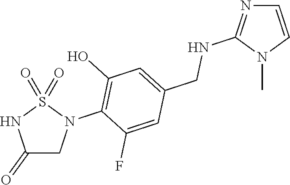 [SMILES: Cn1ccnc1NCc1cc(O)c(N2CC(=O)NS2(=O)=O)c(F)c1]

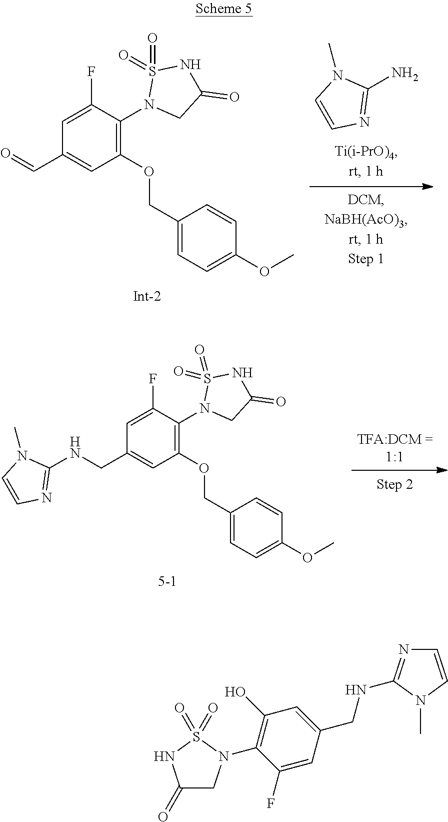

Step 1: To a solution of 3-fluoro-5-[(4-methoxyphenyl)methoxy]-4-(1,1,4-trioxo-1,2,5-thiadiazolidin-2-yl)benzaldehyde (80 mg, 0.2 mmol) and 1-methylimidazol-2-amine (39.4 mg, 0.41 mmol) in dichloromethane (8 mL) was added a solution of Titanium tetraisopropanolate (115.63 mg, 0.41 mmol) at 0° C. The reaction mixture was stirred at room temperature for 2 h. NaBH(AcO)₃ (38.95 mg, 0.41 mmol) was added to the mixture at 0° C. The resulting mixture was stirred at room temperature for additional 30 mins. Upon completion, the reaction mixture was concentrated. The resulting residue was dissolved with DMSO and purified by a reversed-phase column (0.05% NH₄HCO₃ in H₂O and MeCN) to obtain 5-[2-fluoro-6-[(4methoxyphenyl)methoxy]-4-[[(1-methylimidazol-2-yl)amino]methyl]phenyl]-1,1-dioxo-1,2,5-thiadiazolidin-3-one (60 mg, 62.5% yield). MS: m/z: Calc'd for C₂₁H₂₂FN₅O₅S, [M+H]⁺476; Found 476.

Step 2: The title compound was prepared in 30.50% yield as a white solid according to the preparation of EXAMPLE 1 using 16-1 in STEP 2. MS: m/z: Calc'd for C₁₃H₁₄FN₅O₄S, [M+H]+356; Found 356. ¹H NMR (400 MHZ, DMSO-d₆) δ 12.40 (s, 1H), 9.57 (s, 1H), 8.43 (t, J=6.1 Hz, 1H), 7.10 (d, J=2.4 Hz, 1H), 7.05 (d, J=2.4 Hz, 1H), 6.73 (d, J=11.1 Hz, 2H), 4.45 (d, J=6.1 Hz, 2H), 3.98 (s, 2H), 3.54 (s, 3H).

Prep-HPLC purification conditions: Column: SunFire Prep C18 OBD Column, 19*150 mm, 5 μm; Mobile Phase A: Water (0.1% FA), Mobile Phase B: ACN; Flow rate: 20 mL/min; Gradient: 23% B to 45% B in 5 min, 45% B; Wavelength: 210/254 nm

### Example 12: 5-[2-fluoro-6-hydroxy-4-[[(5-methyl-1,3,4-thiadiazol-2-yl) amino]methyl]phenyl]-1,1-dioxo-1,2,5-thiadiazolidin-3-one

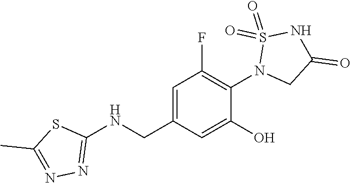 [SMILES: Cc1nnc(NCc2cc(O)c(N3CC(=O)NS3(=O)=O)c(F)c2)s1]

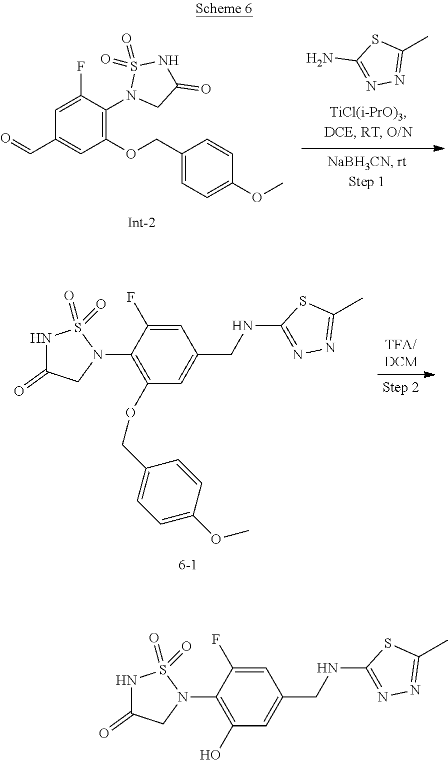

Step 1: To a stirred mixture of 3-fluoro-5-[(4-methoxyphenyl) methoxy]-4-(1,1,4-trioxo-1,2,5-thiadiazolidin-2-yl) benzaldehyde (50 mg, 0.13 mmol) and 5-methyl-1,3,4-thiadiazol-2-amine (14.6 mg, 0.13 mmol) in DCE (5 mL) was added TiCl(i-PrO)₃ (65.93 mg, 0.25 mmol) dropwise. The resulting mixture was stirred at room temperature for 12 h. NaBH₃CN (23.86, 0.38 mmol) was added to the above mixture at 0° C. The resulting mixture was stirred at room temperature for additional 1 h. The reaction mixture was directly purified by a reversed-phase column chromatography (0.05% TFA in H₂O and MeCN) to afford 5-[2-fluoro-6-[(4-methoxyphenyl) methoxy]-4-[[(5-methyl-1,3,4-thiadiazol-2-yl) amino]methyl]phenyl]-1,1-dioxo-1,2,5-thiadiazolidin-3-one (50 mg of TFA salt, 0.10 mmol, 79.90% yield) as an off-white oil. MS: m/z: Calc'd for C₂₀H₂₀FN₅O₅S₂ [M+H]⁺494; Found 494.

Step 2: To a stirred mixture of 5-[2-fluoro-6-[(4-methoxyphenyl) methoxy]-4-[(1,3,4-thiadiazol -2-ylamino) methyl]phenyl]-1,1-dioxo-1,2,5-thiadiazolidin-3-one (45 mg, 0.09 mmol) in DCM (4 mL) was added TFA (4 mL) at room temperature. The resulting mixture was stirred at room temperature for 12 h. After completion of the reaction monitored by LCMS, the mixture was concentrated under reduced pressure. The resulting residue was purified by Prep-HPLC to afford 5-[2-fluoro-6-hydroxy-4-[[(5-methyl-1,3,4-thiadiazol-2-yl) amino]methyl]phenyl]-1,1-dioxo -1,2,5-thiadiazolidin-3-one (6.6 mg, 0.01 mmol, 18.17% yield) as a white solid. MS: m/z: Calc'd for C₁₂H₁₂FN₅O₄S₂ [M+H]⁺374; Found 374. ¹H NMR (400 MHZ, DMSO-d₆+D₂O) δ 7.58-7.50 (m, 1H), 6.82-6.48 (m, 5H), 4.38 (s, 2H), 3.94 (s, 2H).

Prep-HPLC purification conditions: Column: XBridge Prep OBD C18 Column, 19*250 mm, 5 μm; Mobile Phase A: Water (10 mmol/L NH₄HCO₃), Mobile Phase B: ACN; Flow rate: 25 mL/min; Gradient: 28% B to 48% B in 6 min, 48% B; Wavelength: 254/210 nm.

### Example 13: 5-[2-fluoro-6-hydroxy-4-[[(4-methylthiazol-2-yl) amino]methyl]phenyl]-1,1-dioxo-1,2,5-thiadiazolidin-3-one

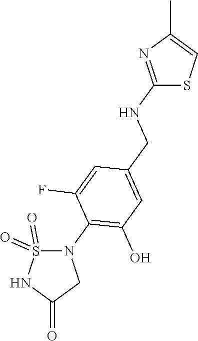 [SMILES: Cc1csc(NCc2cc(O)c(N3CC(=O)NS3(=O)=O)c(F)c2)n1]

The title compound was prepared in 14.16% overall yield as a white solid according to the preparation of EXAMPLE 9 using 4-methylthiazol-2-amine in STEP 1. After the addition of NaBH₃CN, the reaction was performed at 80° C. instead of room temperature. MS: m/z: Calc'd for C₁₃H₁₃FN₄O₄S₂, [M+H]⁺373; Found 373. 1H NMR (400 MHZ, DMSO-d₆) δ 6.71-6.58 (m, 2H), 6.22 (d, J=1.3 Hz, 1H), 4.32 (s, 2H), 3.99 (s, 2H), 2.07 (s, 3H).

Prep-HPLC purification conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 55% B to 75% B in 8 min, 75% B; Wavelength: 254/210 nm.

### Example 14: 5-[2-fluoro-6-[(4-methoxyphenyl)methoxy]-4-[[(3-methylisothiazol-5-yl)amino]methyl]phenyl]-1,1-dioxo-1,2,5-thiadiazolidin-3-one

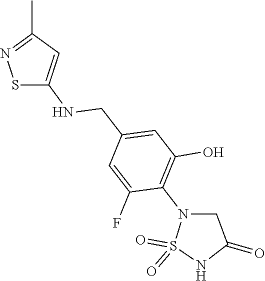 [SMILES: Cc1cc(NCc2cc(O)c(N3CC(=O)NS3(=O)=O)c(F)c2)sn1]

The title compound was prepared in 7.31% overall yield as a white solid according to the preparation of EXAMPLE 4 using 3-methylisothiazol-5-amine in STEP 1. MS: m/z: Calc'd for C₁₃H₁₃FN₄O₄S₂, [M+H]⁺373; Found 373. ¹H NMR (400 MHZ, DMSO-d₆) δ 10.37 (s, 1H), 7.80 (s, 1H), 6.78-6.62 (m, 2H), 6.03 (s, 1H), 4.34 (s, 2H), 4.23 (s, 2H), 2.16 (s, 3H).

Purification conditions: the compound was purified by a reversed-phase column chromatography (0.05% NH₄HCO₃ in H₂O and MeCN).

### Example 15: 5-[2-fluoro-6-hydroxy-4-[[(3-methyl-1H-pyrazol-5-yl)amino]methyl]phenyl]-1,1-dioxo-1,2,5-thiadiazolidin-3-one

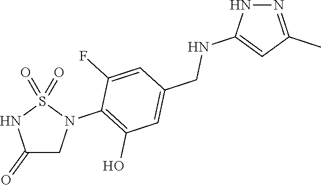 [SMILES: Cc1cc(NCc2cc(O)c(N3CC(=O)NS3(=O)=O)c(F)c2)[nH]n1]

The title compound was prepared in 12.20% overall yield as a white solid according to the preparation of EXAMPLE 9 using 5-methyl-1H-pyrazol-3-amine in STEP 1. MS: m/z: Calc'd for C₁₃H₁₄FN₅O₄S, [M+H]⁺356; Found 356. ¹H NMR (400 MHZ, DMSO-d₆) δ 13.8-12.8 (m, 1H), 9.60 (s, 1H), 7.47 (s, 1H), 6.63 (d, J=12.5 Hz, 2H), 5.60 (s, 1H), 4.22 (s, 2H), 3.99 (s, 2H), 2.19 (s, 3H).

Prep-HPLC purification conditions: Column: SunFire Prep C18 OBD Column, 19*150 mm, 5 μm; Mobile Phase A: Water (0.1% FA), Mobile Phase B: ACN; Flow rate: 60 mL/min; Gradient: 40% B to 60% B in 6.58 min, 60% B; Wavelength: 254/210 nm.

### Compound Examples Prepared by the Procedures above are listed in Table 1

### Examples of Prepared Compounds

TABLE 1

| Compound | | |
|---|---|---|
| number | Structure | Chemical name |
| EXAMPLE 1 | 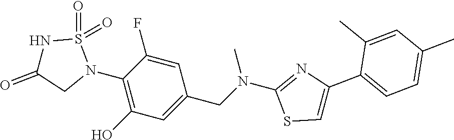 [SMILES: Cc1ccc(-c2csc(N(C)Cc3cc(O)c(N4CC(=O)NS4(=O)=O)c(F)c3)n2)c(C)c1] | 5-(4-(((4-(2,4- dimethylphenyl)thiazol-2- yl)(methyl)amino)methyl)-2- fluoro-6-hydroxyphenyl)- 1,2,5-thiadiazolidin-3-one 1,1-dioxide |
| EXAMPLE 2 | 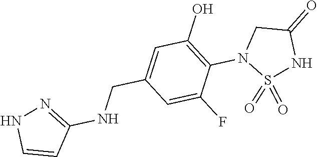 [SMILES: O=C1CN(c2c(O)cc(CNc3cc[nH]n3)cc2F)S(=O)(=O)N1] | 5-(4-(((1H-pyrazol-3- yl)amino)methyl)-2-fluoro-6- hydroxyphenyl)-1,2,5- thiadiazolidin-3-one 1,1- dioxide |
| EXAMPLE 3 | 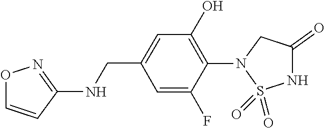 [SMILES: O=C1CN(c2c(O)cc(CNc3ccon3)cc2F)S(=O)(=O)N1] | 5-(2-fluoro-6-hydroxy-4- ((isoxazol-3- ylamino)methyl)phenyl)- 1,2,5-thiadiazolidin-3-one 1,1- dioxide |
| EXAMPLE 4 | 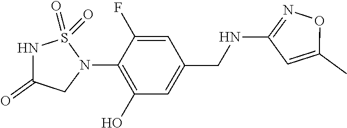 [SMILES: Cc1cc(NCc2cc(O)c(N3CC(=O)NS3(=O)=O)c(F)c2)no1] | 5-(2-fluoro-6-hydroxy-4-(((5- methylisoxazol-3- yl)amino)methyl)phenyl)- 1,2,5-thiadiazolidin-3-one 1,1- dioxide |
| EXAMPLE 5 | 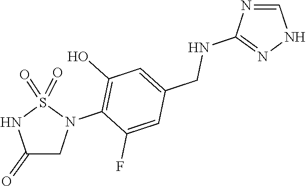 [SMILES: O=C1CN(c2c(O)cc(CNc3nc[nH]n3)cc2F)S(=O)(=O)N1] | 5-(4-(((1H-1,2,4-triazol-3- yl)amino)methyl)-2-fluoro-6- hydroxyphenyl)-1,2,5- thiadiazolidin-3-one 1,1- dioxide |
| EXAMPLE 6 | 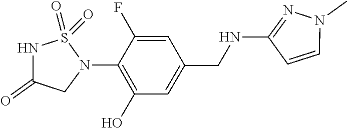 [SMILES: Cn1ccc(NCc2cc(O)c(N3CC(=O)NS3(=O)=O)c(F)c2)n1] | 5-(2-fluoro-6-hydroxy-4-(((1- methyl-1H-pyrazol-3- yl)amino)methyl)phenyl)- 1,2,5-thiadiazolidin-3-one 1,1- dioxide |
| EXAMPLE 7 | 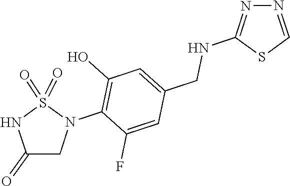 [SMILES: O=C1CN(c2c(O)cc(CNc3nncs3)cc2F)S(=O)(=O)N1] | 5-(4-(((1,3,4-thiadiazol-2- yl)amino)methyl)-2-fluoro-6- hydroxyphenyl)-1,2,5- thiadiazolidin-3-one 1,1- dioxide |
| EXAMPLE 8 | 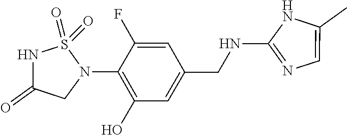 [SMILES: Cc1cnc(NCc2cc(O)c(N3CC(=O)NS3(=O)=O)c(F)c2)[nH]1] | 5-(2-fluoro-6-hydroxy-4-(((5- methyl-1H-imidazol-2- yl)amino)methyl)phenyl)- 1,2,5-thiadiazolidin-3-one 1,1- dioxide |
| EXAMPLE 9 | 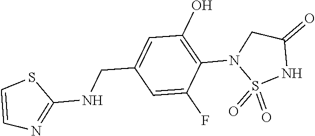 [SMILES: O=C1CN(c2c(O)cc(CNc3nccs3)cc2F)S(=O)(=O)N1] | 5-(2-fluoro-6-hydroxy-4- ((thiazol-2- ylamino)methyl)phenyl)- 1,2,5-thiadiazolidin-3-one 1,1- dioxide |
| EXAMPLE 10 | 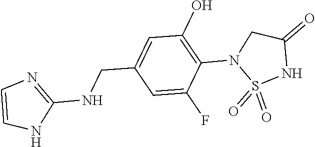 [SMILES: O=C1CN(c2c(O)cc(CNc3ncc[nH]3)cc2F)S(=O)(=O)N1] | 5-(4-(((1H-imidazol-2- yl)amino)methyl)-2-fluoro-6- hydroxyphenyl)-1,2,5- thiadiazolidin-3-one 1,1- dioxide |
| EXAMPLE 11 | 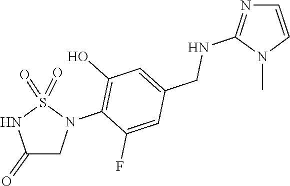 [SMILES: Cn1ccnc1NCc1cc(O)c(N2CC(=O)NS2(=O)=O)c(F)c1] | 5-(2-fluoro-6-hydroxy-4-(((1- methyl-1H-imidazol-2- yl)amino)methyl)phenyl)- 1,2,5-thiadiazolidin-3-one 1,1- dioxide |
| EXAMPLE 12 | 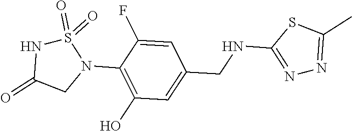 [SMILES: Cc1nnc(NCc2cc(O)c(N3CC(=O)NS3(=O)=O)c(F)c2)s1] | 5-(2-fluoro-6-hydroxy-4-(((5- methyl-1,3,4-thiadiazol-2- yl)amino)methyl)phenyl)- 1,2,5-thiadiazolidin-3-one 1,1- dioxide |
| EXAMPLE 13 | 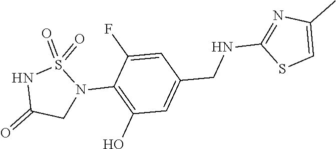 [SMILES: Cc1csc(NCc2cc(O)c(N3CC(=O)NS3(=O)=O)c(F)c2)n1] | 5-(2-fluoro-6-hydroxy-4-(((4- methylthiazol-2- yl)amino)methyl)phenyl)- 1,2,5-thiadiazolidin-3-one 1,1- dioxide |
| EXAMPLE 14 | 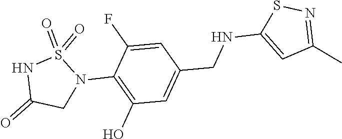 [SMILES: Cc1cc(NCc2cc(O)c(N3CC(=O)NS3(=O)=O)c(F)c2)sn1] | 5-(2-fluoro-6-hydroxy-4-(((3- methylisothiazol-5- yl)amino)methyl)phenyl)- 1,2,5-thiadiazolidin-3-one 1,1- dioxide |
| EXAMPLE 15 | 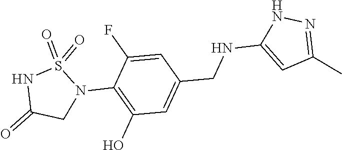 [SMILES: Cc1cc(NCc2cc(O)c(N3CC(=O)NS3(=O)=O)c(F)c2)[nH]n1] | 5-(2-fluoro-6-hydroxy-4-(((3- methyl-1H-pyrazol-5- yl)amino)methyl)phenyl)- 1,2,5-thiadiazolidin-3-one 1,1- dioxide |

### Biological Assays

The pharmacological properties of the compounds of this invention may be confirmed by a number of biological assays known in the art. The exemplified biological assays which follow, have been carried out with compounds of the invention.

### Assays

A PhosphoSens® kinase assay was performed as described by the vendor (AssayQuant Technologies, Marlborough, MA). Briefly, 1000× solutions of compounds were prepared in DMSO via serial dilution of the 10 mM DMSO stocks using 3-fold intervals in a 384-well reagent plate. 50 nL of the compound dilution series was then added to the corresponding wells of a 384-well assay plate. 40 mL of 1.25× substrate (AQT0264) in 1× assay buffer (50 mM HEPES pH 7.5, 500 μM EGTA, 10 nM MgCl₂, 0.01% Brij-35, 1% Glycerol, 1 mM DTT, and 0.2 mg/mL BSA) was transferred to each well of the assay plate to achieve a final substrate concentration of 20 μM. Finally, 10 mL of 5× PTPN2 enzyme stock was added to each well of the assay plate for a final enzyme concentration of 150 pM. Reaction progress curves were collected by sampling fluorescence intensity at the excitation wavelength 360 nm (λₑₓ360) and emission wavelength 480 nm (λₑₘ480) every 71 seconds for one hour using a Synergy H4 plate reader (BioTek Instruments/Agilent Technologies, Winooski, VT) at room temperature.

### Phosphotase Activity Assay Using DIFMUP as Substrate

The PTPN2 biochemical assay was performed as follows, a 5× stock solution of human PTPN2 (SRP5075, MilliporeSigma, Burlington, MA) and a 1.25× stock solution of DiFMUP (D6567, ThermoFisher Scientific, Waltham, MA), were prepared in 1× reaction buffer consisting of 50 mM HEPES, pH 7.4, 1 mM EDTA, 150 mM NaCl, 0.2 mg/mL BSA, 100 U/mL catalase and 10 mM DTT. 40 mL of the DiFMUP substrate solution, for a final concentration of 25 mM DiFMUP substrate, was added to a Corning 3574 384-well, white, non-binding surface microtiter plate containing 0.05 mL of serially diluted test compounds prepared in DMSO. The reactions were started with the addition of 10 mL of the enzyme solution, for a final PTPN2 concentration of 0.15 nM, and monitored every 105 seconds for 60 minutes at λ_{EX} 360/λ_{EM} 460 in a BioTek Synergy HTX plate reader (Agilent Technologies, Santa Clara, CA) at room temperature. The initial linear portions of the progress curves were fit according to a linear equation to yield the slopes and converted to % inhibition based on a value of 100% activity for the no inhibitor treated control. IC₅₀ values of each compound were obtained by fitting the % inhibition-compound concentration curves using Dotmatics software (Dotmatics, Bishops Stortford, Hertfordshire, England).

### Cell Proliferation Assay Protocol

B16-F10 cells (ATCC, Manassas, VA, #CRL-6475) were cultured in DMEM growth medium (ThermoFisher Scientific, Waltham, MA, #11995-040) supplemented with 10% heat inactivated FBS (ThermoFisher Scientific, #16140-071) and 1% pen/strep (ThermoFisher Scientific, #15140-122). The cells were seeded into two white opaque 384-well tissue culture treated microplates (PerkinElmer, Waltham, MA, #6007688) at a density of 100 cells/well in 20 uL total volume and incubated overnight at 37 C and 5% CO2. 30 nL of compounds dissolved in DMSO were then transferred from a source plate into target wells with the Echo650 acoustic liquid handler (Beckman Coulter, Indianapolis, IN). Negative control wells received 30 nL of DMSO only (0.15% final concentration). Plates were returned to the incubator for 1 hour and then cells treated with either 5 uL of growth medium or 5 uL of growth medium containing 50 ng/ml of recombinant mouse IFN-gamma protein (R&D Systems, Minneapolis, MN, #485-MI/CF, 10 ng/mL final concentration) using the Assist automated pipetting platform (INTEGRA Biosciences, Hudson, NH). Plates were incubated at 37C for 4 days and cell proliferation assayed with the CellTiter-Glo reagent (Promega, Madison, WI, #G7573, 25 uL per well). Luminescence signal intensity was collected with the EnVision 2105 plate reader (PerkinElmer) 15 minutes after CellTiter-Glo reagent addition and analyzed with the Dotmatics software platform to calculate compound IC50 values. Off-target compound mediated cytotoxicity was identified by checking for growth inhibition in the absence of IFNg.

### Phospho-STAT1 Assay Protocol

B16-F10 cells (ATCC, Manassas, VA, #CRL-6475) were cultured in DMEM growth medium (ThermoFisher Scientific, Waltham, MA, #11995-040) supplemented with 10% heat inactivated FBS (ThermoFisher Scientific, #16140-071) and 1% pen/strep (ThermoFisher Scientific, #15140-122). The cells were seeded into a white opaque 384-well tissue culture treated microplate (PerkinElmer, Waltham, MA, #6007688) at a density of 10,000 cells/well in 20 uL total volume and incubated overnight at 37 C and 5% CO2. 30 nL of compounds dissolved in DMSO were then transferred from a source plate into target wells with the Echo650 acoustic liquid handler (Beckman Coulter, Indianapolis, IN). Negative control wells received 30 nL of DMSO only (0.15% final concentration). Plates were returned to the incubator for 1 hour and then cells treated with either 5 uL of growth medium or 5 uL of growth medium containing 500 ng/ml of recombinant mouse IFN-gamma protein (R&D Systems, Minneapolis, MN, #485-MI/CF, 100 ng/mL final concentration) using the Assist automated pipetting platform (INTEGRA Biosciences, Hudson, NH). Plates were incubated at 37 C for 1 hour and assayed for phosphorylated STAT1 protein levels with the phospho-STAT1 (Tyr701) HTRF kit (Cisbio, Bedford, MA, #63ADK026PEH) according to manufacturer's instructions. HTRF signal intensity was collected with the EnVision 2105 plate reader (PerkinElmer) 24 hours later and analyzed with the Dotmatics software platform to calculate compound IC50 values.

### Biological Assay Data

Table 2 is a summary of Biological Assay data for Examples/Embodiments Prepared. For IC50 data, High DDT concentration and/or DiFMUP substrate assays were used; a skilled artisan may use either assay. A row or column with a double asterisk indicates that one IC50 value or embodiment has been provided.

TABLE 2

| | | | PTPN2 | pSTAT1 | PTPN2 |
|---|---|---|---|---|---|
| | | PTPN2 | BCHEM | HTRF | Prolif 5d |
| | | BCHEM | DIFMUP | B16 | B16F10 |
| Compound | | IC50 | IC50 | EC50 | EC50 |
| Number | Iupac Name | (uM) | (uM) | (uM) | (uM) |
| EXAMPLE | 5-(4-(((4-(2,4- | 0.94 | 0.72 | >15 | >15 |
| 1 | dimethylphenyl)thiazol-2- | | | | |
| | yl)(methyl)amino)methyl)- | | | | |
| | 2-fluoro-6- | | | | |
| | hydroxyphenyl)-1,2,5- | | | | |
| | thiadiazolidin-3-one 1,1- | | | | |
| | dioxide | | | | |
| EXAMPLE | 5-(4-(((1H-pyrazol-3- | ** | 0.02 | >15 | >15 |
| 2 | yl)amino)methyl)-2- | | | | |
| | fluoro-6-hydroxyphenyl)- | | | | |
| | 1,2,5-thiadiazolidin-3-one | | | | |
| | 1,1-dioxide | | | | |
| EXAMPLE | 5-(2-fluoro-6-hydroxy-4- | ** | 0.02 | 0.03 | 1.12 |
| 3 | ((isoxazol-3- | | | | |
| | ylamino)methyl)phenyl)- | | | | |
| | 1,2,5-thiadiazolidin-3-one | | | | |
| | 1,1-dioxide | | | | |
| EXAMPLE | 5-(2-fluoro-6-hydroxy-4- | 0.02 | 0.03 | 0.35 | 1.03 |
| 4 | (((5-methylisoxazol-3- | | | | |
| | yl)amino)methyl)phenyl)- | | | | |
| | 1,2,5-thiadiazolidin-3-one | | | | |
| | 1,1-dioxide | | | | |
| EXAMPLE | 5-(4-(((1H-1,2,4-triazol-3- | ** | 0.04 | 0.93 | 1.69 |
| 5 | yl)amino)methyl)-2- | | | | |
| | fluoro-6-hydroxyphenyl)- | | | | |
| | 1,2,5-thiadiazolidin-3-one | | | | |
| | 1,1-dioxide | | | | |
| EXAMPLE | 5-(2-fluoro-6-hydroxy-4- | ** | 0.01 | 6.85 | 0.82 |
| 6 | (((1-methyl-1H-pyrazol-3- | | | | |
| | yl)amino)methyl)phenyl)- | | | | |
| | 1,2,5-thiadiazolidin-3-one | | | | |
| | 1,1-dioxide | | | | |
| EXAMPLE | 5-(4-(((1,3,4-thiadiazol-2- | ** | 0.01 | 0.15 | 0.46 |
| 7 | yl)amino)methyl)-2- | | | | |
| | fluoro-6-hydroxyphenyl)- | | | | |
| | 1,2,5-thiadiazolidin-3-one | | | | |
| | 1,1-dioxide | | | | |
| EXAMPLE | 5-(2-fluoro-6-hydroxy-4- | ** | 0.16 | 4.65 | 5.9 |
| 8 | (((5-methyl-1H-imidazol-2- | | | | |
| | yl)amino)methyl)phenyl)- | | | | |
| | 1,2,5-thiadiazolidin-3-one | | | | |
| | 1,1-dioxide | | | | |
| EXAMPLE | 5-(2-fluoro-6-hydroxy-4- | ** | 0.003 | 1.43 | 2.3 |
| 9 | ((thiazol-2- | | | | |
| | ylamino)methyl)phenyl)- | | | | |
| | 1,2,5-thiadiazolidin-3-one | | | | |
| | 1,1-dioxide | | | | |
| EXAMPLE | 5-(4-(((1H-imidazol-2- | ** | 0.006 | 0.09 | 0.18 |
| 10 | yl)amino)methyl)-2- | | | | |
| | fluoro-6-hydroxyphenyl)- | | | | |
| | 1,2,5-thiadiazolidin-3-one | | | | |
| | 1,1-dioxide | | | | |
| EXAMPLE | 5-(2-fluoro-6-hydroxy-4- | ** | 0.01 | 0.21 | 0.16 |
| 11 | (((1-methyl-1H-imidazol-2- | | | | |
| | yl)amino)methyl)phenyl)- | | | | |
| | 1,2,5-thiadiazolidin-3-one | | | | |
| | 1,1-dioxide | | | | |
| EXAMPLE | 5-(2-fluoro-6-hydroxy-4- | ** | 0.01 | 0.23 | 0.56 |
| 12 | (((5-methyl-1,3,4- | | | | |
| | thiadiazol-2- | | | | |
| | yl)amino)methyl)phenyl)- | | | | |
| | 1,2,5-thiadiazolidin-3-one | | | | |
| | 1,1-dioxide | | | | |
| EXAMPLE | 5-(2-fluoro-6-hydroxy-4- | ** | 0.01 | 0.23 | 1.87 |
| 13 | (((4-methylthiazol-2- | | | | |
| | yl)amino)methyl)phenyl)- | | | | |
| | 1,2,5-thiadiazolidin-3-one | | | | |
| | 1,1-dioxide | | | | |
| EXAMPLE | 5-(2-fluoro-6-hydroxy-4- | ** | 0.02 | 0.69 | 2.57 |
| 14 | (((3-methylisothiazol-5- | | | | |
| | yl)amino)methyl)phenyl)- | | | | |
| | 1,2,5-thiadiazolidin-3-one | | | | |
| | 1,1-dioxide | | | | |
| EXAMPLE | 5-(2-fluoro-6-hydroxy-4- | ** | 0.004 | 0.63 | 0.38 |
| 15 | (((3-methyl-1H-pyrazol-5- | | | | |
| | yl)amino)methyl)phenyl)- | | | | |
| | 1,2,5-thiadiazolidin-3-one | | | | |
| | 1,1-dioxide | | | | |

What is claimed is:

1. A compound having the following structure of Formula I: 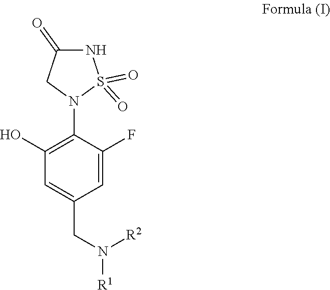
wherein, independently for each occurrence:
R¹ is selected from the group consisting of: 1H-1,2,4-triazol-3-yl, 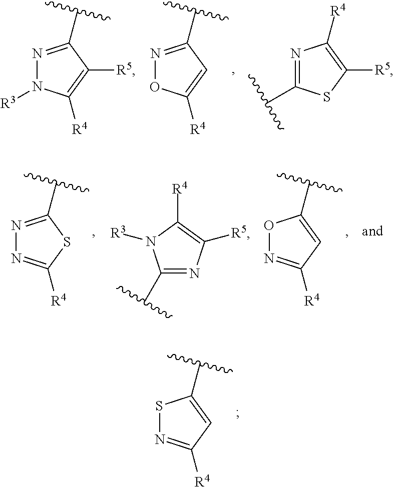
R² is selected from the group consisting of: —H, alkyl and substituted alkyl;
R³ is selected from the group consisting of: —- H, alkyl and substituted alkyl;
R⁴ is selected from the group consisting of: —H, alkyl, substituted alkyl, branched alkyl, alkoxy, halogen, cyano, amine, hydroxy, aryl and substituted aryl;
R⁵ is selected from the group consisting of: —H, alkyl, substituted alkyl, branched alkyl, alkoxy, halogen, cyano, amine, hydroxy, aryl and substituted aryl.

2. The compound according to claim 1, wherein:
R¹ is selected from the group consisting of: 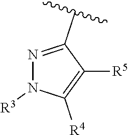
R³ is selected from the group consisting of: —H and —CH₃;
R⁴ is —H and —CH₃.

3. The compound according to claim 1, wherein:
R¹ is 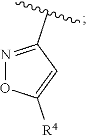
R⁴ is selected from the group consisting of: —H and —CH₃.

4. The compound according to claim 1, wherein:
R¹ is 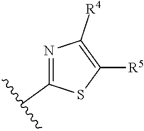
R⁴ is selected from the group consisting of: —H, —CH₃, and 2,4-dimethylphenyl;
R⁵ is selected from the group consisting of: —H and —CH₃.

5. The compound according to claim 1, wherein:
R¹ is selected from the group consisting of: 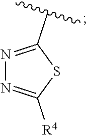
R⁴ is selected from the group consisting of: —H and —CH₃.

6. The compound according to claim 1, wherein:
R¹ is 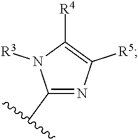
R³ is selected from the group consisting of: —H and —CH₃
R⁴ is —H and —CH₃;
R⁵ is —H and —CH₃.

7. A compound selected from the group consisting of:
5-(4-(((4-(2,4-dimethylphenyl)thiazol-2-yl)(methyl)amino)methyl)-2-fluoro-6-hydroxyphenyl)-1,2,5-thiadiazolidin-3-one 1,1-dioxide;
5-(4-(((1H-pyrazol-3-yl)amino)methyl)-2-fluoro-6-hydroxyphenyl)-1,2,5-thiadiazolidin -3-one 1,1-dioxide;
5-(2-fluoro-6-hydroxy-4-((isoxazol-3-ylamino)methyl)phenyl)-1,2,5-thiadiazolidin-3-one 1,1-dioxide;
5-(2-fluoro-6-hydroxy-4-(((5-methylisoxazol-3-yl)amino)methyl)phenyl)-1,2,5-thiadiazolidin-3-one 1,1-dioxide;
5-(4-(((1H-1,2,4-triazol-3-yl)amino)methyl)-2-fluoro-6-hydroxyphenyl)-1,2,5-thiadiazolidin-3-one 1,1-dioxide;
5-(2-fluoro-6-hydroxy-4-(((1-methyl-1H-pyrazol-3-yl)amino)methyl)phenyl)-1,2,5-thiadiazolidin-3-one 1,1-dioxide;
5-(4-(((1,3,4-thiadiazol-2-yl)amino)methyl)-2-fluoro-6-hydroxyphenyl)-1,2,5-thiadiazolidin-3-one 1,1-dioxide;
5-(2-fluoro-6-hydroxy-4-(((5-methyl-1H-imidazol-2-yl)amino)methyl)phenyl)-1,2,5-thiadiazolidin-3-one 1,1-dioxide;
5-(2-fluoro-6-hydroxy-4-((thiazol-2-ylamino)methyl)phenyl)-1,2,5-thiadiazolidin-3-one 1,1-dioxide;
5-(4-(((1H-imidazol-2-yl)amino)methyl)-2-fluoro-6-hydroxyphenyl)-1,2,5-thiadiazolidin -3-one 1,1-dioxide;
5-(2-fluoro-6-hydroxy-4-(((1-methyl-1H-imidazol-2-yl)amino)methyl)phenyl)-1,2,5-thiadiazolidin-3-one 1,1-dioxide;
5-(2-fluoro-6-hydroxy-4-(((5-methyl-1,3,4-thiadiazol-2-yl)amino)methyl)phenyl)-1,2,5-thiadiazolidin-3-one 1,1-dioxide;
5-(2-fluoro-6-hydroxy-4-(((4-methylthiazol-2-yl)amino)methyl)phenyl)-1,2,5-thiadiazolidin-3-one 1,1-dioxide;
5-(2-fluoro-6-hydroxy-4-(((3-methylisothiazol-5-yl)amino)methyl)phenyl)-1,2,5-thiadiazolidin-3-one 1,1-dioxide;
5-(2-fluoro-6-hydroxy-4-(((3-methyl-1H-pyrazol-5-yl)amino)methyl)phenyl)-1,2,5-thiadiazolidin-3-one 1,1-dioxide;
or pharmaceutically acceptable salts thereof.

8. A pharmaceutical composition comprising a compound of Formula (I) according to claim 1, or a pharmaceutically acceptable salt thereof, and at least one pharmaceutically acceptable carrier.

9. A method for treating cancer comprising administering to said patient a therapeutically effective amount of a compound of Formula (I) according to claim 1 or a pharmaceutically acceptable salt thereof wherein the cancer/disease is selected from: human cancers, carcinomas, sarcomas, adenocarcinomas, papillary adenocarcinomas, lymphomas, leukemias, melanomas, solid lymphoid cancers, kidney cancer, breast cancer, lung cancer, bladder cancer, colon cancer, ovarian cancer, prostate cancer, pancreatic cancer, stomach cancer, brain cancer, head and neck cancer, skin cancer, uterine, testicular, glioma, esophagus, liver cancer, including hepatocarcinoma, lymphoma, including B-acute lymphoblastic lymphoma, non-Hodgkin's lymphomas, Burkitt's lymphoma, Small lymphomas, Hodgkin's lymphoma, leukemia, and multiple myeloma.

10. A method of treating cancer in a patient in need thereof, comprising administering to the patient an effective amount of a compound of claim 1 in combination with an additional therapeutic agent.

11. The method of claim 10 wherein the additional therapeutic agent is an immunotherapeutic agent.

12. The method of claim 11 wherein the immunotherapeutic agent is selected from the group consisting of an anti-PD-1 antibody, an anti-PD-L1 antibody, and an anti-CTLA-4 antibody.

13. A method of treating cancer in a patient in need thereof, said method comprising administering to the patient an effective amount of a pharmaceutically acceptable composition of claim 1.

14. The method of claim 1 wherein the method of treating cancer is selected from: radiation, surgery, chemotherapy, or administration of a biologic drug.

15. The method of claim 14 wherein the method of treating cancer further comprises the administration of a biologic drug and the biologic drug is a drug that stimulates the immune system.

16. The method of claim 15, wherein the method further comprises administering to the subject an inhibitor of DGKα and/or DGKζ, an antagonist of the PD1/PD-L1 axis and an antagonist of CTLA4.
